(19) United States
(12) Patent Application Publication
(10) Pub. No.: US 2026/0199524 A1
(43) Pub. Date: Jul. 16, 2026

(54) VARIANTS OF SIRTUIN 6 FOR THE TREATMENT OF NON-ALCOHOLIC FATTY LIVER DISEASE

(71) Applicant: GENFLOW BIOSCIENCES SRL, Charleroi (BE)

(72) Inventors: Manlio VINCIGUERRA, Liverpool (GB); Eric LEIRE, Watermael-Boitsfort (BE)

(73) Assignee: GENFLOW BIOSCIENCES SRL, Charleroi (BE)

(21) Appl. No.: 19/136,424

(22) PCT Filed: Dec. 8, 2023
(86) PCT No.: PCT/EP2023/084840

§ 371 Date: Jun. 6, 2025

Related U.S. Application Data
(60) Provisional application No. 63/431,146, filed on Dec. 8, 2022.

(51) Int. Cl. A61K 48/00; A61K 38/00; A61P 1/16; C12N 9/10; C12N 15/86

(52) U.S. Cl. CPC A61K 48/005; A61P 1/16; C12N 9/1029; C12N 15/86; A61K 38/00; C12N 2750/14143; C12Y 203/01

(57) ABSTRACT

The present invention relates to variants of sirtuin 6 for the treatment of non-alcoholic fatty liver disease.

### FIELD OF INVENTION

The present invention relates to a prophylactic and/or therapeutic composition for non-alcoholic fatty liver disease (NAFLD), particularly non-alcoholic steatohepatitis (NASH), which contains a variant of SIRT6.

### BACKGROUND OF INVENTION

The accumulation of fat in the liver (steatosis) is classically promoted by excessive alcohol consumption. Non-alcoholic fatty liver disease (NAFLD) is the generic term for the excessive accumulation of fat in the liver not related to the excessive consumption of alcoholic beverages.

Today, non-alcoholic fatty liver disease affects approximately 20% of the general population. Steatosis is most often isolated (in about 80% of cases). It is then a benign situation with a very low risk of complications. In the remaining 20% of cases, steatosis is responsible for liver cell damage (ballooning of the hepatocytes) and inflammation of the liver parenchyma: this is steatohepatitis or NASH (for “Non-Alcoholic SteatoHepatitis”).

Steatohepatitis represents the aggressive form of the disease because it promotes the accumulation of hepatic fibrosis in the liver. This is graded in five stages (0 to 4), stage 4 corresponding to cirrhosis. Among all people with steatosis, less than 5% have pre-cirrhotic fibrosis and 1% have cirrhosis. These percentages may seem small, but given the frequency of steatosis, they ultimately represent a significant number of people.

### SUMMARY

This invention thus relates to an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1 for use in the prevention and/or the treatment of non-alcoholic fatty liver disease (NAFLD).

In one embodiment, the nucleic acid molecule is of sequence selected in the group comprising or consisting of SEQ ID NO: 6, SEQ ID NO: 7 and SEQ ID NO: 8.

This invention also relates to an isolated polypeptide encoded by a nucleic acid molecule as described hereinabove for use in the prevention and/or the treatment of NAFLD.

In one embodiment, the polypeptide is of sequence selected in the group comprising or consisting of SEQ ID NO: 2, SEQ ID NO: 3 and SEQ ID NO: 4.

This invention further relates to a vector comprising the isolated nucleic acid molecule as described hereinabove for use in the prevention and/or the treatment of NAFLD.

In one embodiment, the vector is a viral vector, in particular an adeno-associated viral vector (AAV), an exosome-associated AAV vector (exo-AAV), an adenoviral vector, a retroviral vector, or a herpes virus vector.

This invention further relates to a suspension comprising a vector as described hereinabove for use in the prevention and/or the treatment of NAFLD.

This invention further relates to a cell expressing the polypeptide for use as described hereinabove, the cell being preferably transfected with an isolated nucleic acid molecule for use as described hereinabove, or a vector for use as described hereinabove, for use in the prevention and/or the treatment of NAFLD.

Another object of the invention is a pharmaceutical composition comprising (i) an isolated nucleic acid molecule as described hereinabove, or an isolated polypeptide as described hereinabove, or a vector as described hereinabove, and (ii) a pharmaceutically acceptable excipient, for use in the prevention and/or treatment of NAFLD.

Another object of the invention is the isolated acid nucleic molecule for use as described hereinabove, the isolated polypeptide for use as described hereinabove, the vector for use as described hereinabove, the suspension for use as described hereinabove, the cell for use as described hereinabove or the pharmaceutical composition for use as described hereinabove, for the prevention and/or treatment of Stage 1 or Stage 2 of NAFLD.

In one embodiment, the isolated acid nucleic molecule, isolated polypeptide, vector, suspension, cell or pharmaceutical composition is for the prevention and/or treatment of Stage 1 of NAFLD.

In one embodiment, the isolated acid nucleic molecule, isolated polypeptide, vector, suspension, cell or pharmaceutical composition is for the prevention and/or treatment of Stage 2 of NAFLD.

In one embodiment, NASH is Stage 1. In another embodiment, NASH is Stage 2. In one embodiment, NASH is Stage 3.

This invention also relates to a method of preventing and/or treating non-alcoholic fatty liver disease (NAFLD) comprising administering to a patient in need thereof a therapeutically effective amount of an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1, or of an isolated polypeptide encoded by the same or of a pharmaceutical composition comprising the same.

In one embodiment, NAFLD is Stage 1 or 2.

In one embodiment, the isolated nucleic acid molecule is comprised in a vector, preferably a viral vector, more preferably an adeno-associated viral vector (AAV), an exosome-associated AAV vector (exo-AAV), an adenoviral vector, a retroviral vector, or a herpes virus vector.

In one embodiment, the method of the invention further comprises administering to the patient another therapeutic agent.

A further object of this invention is the use of an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1 for the manufacture of a pharmaceutical composition for the prevention and/or treatment of non-alcoholic fatty liver disease (NAFLD).

In one embodiment, NAFLD is Stage 1 or 2.

### Definitions

In the present invention, the following terms have the following meanings:

“About”, when preceding a figure, means plus or less 10% of the value of said figure. It is to be understood that the value to which the term “about” refers is itself also specifically, and preferably, disclosed.

“Comprise” is intended to mean “contain”, “encompass” and “include”. In some embodiments, the term “comprise” also encompasses the term “consist of”.

“Sirtuin 6” also referred to as “SIRT6”, is intended to refer to the polypeptide with the Entrez Gene number 51548, and also non-limitatively relates to the NAD Dependent Protein Deacetylase Sirtuin-6, Regulatory Protein SIR2 Homolog 6, SIR2-Like Protein 6, SIR2L6, Sirtuin (Silent Mating Type Information Regulation 2, S. Cerevisiae, Homolog) 6, Sirtuin (Silent Mating Type Information Regulation 2 Homolog) 6, Sir2-Related Protein Type 6, Sirtuin Type 6 and EC 2.3.1.286.

“Isolated” refers to a nucleic acid molecule or polypeptide a that is removed from the initial biological context that has allowed to generate this nucleic acid molecule or polypeptide. In practice the biological context comprises at least a cell, or one or more enzyme(s).

“Nucleic acid”, also referred to as “polynucleotide”, refers to any polyribonucleotide or polydeoxyribonucleotide, which may be unmodified RNA or DNA or modified RNA or DNA. “Nucleic acid” or “polynucleotide” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acid residues other than the 20 gene-encoded amino acid residues.

“Suspension” refers to a liquid mixture in which the active principle, such as the nucleic acid molecules, polypeptides, or vectors according to the invention, is/are floating in a liquid medium.

“Treating” or “treatment” or “alleviation” refers to both therapeutic treatment and prophylactic or preventative measures, wherein the object is to prevent or slow down (lessen) the targeted pathologic condition or disorder, in particular a liver-related disease, particularly NAFLD, NASH or cirrhosis. Those in need of treatment include those already with said disorder as well as those prone to develop the disorder or those in whom the disorder is to be prevented. An individual is successfully “treated” for a liver-related disease, particularly NAFLD, NASH or cirrhosis, if, after receiving a therapeutic amount of the active principle, in particular the nucleic acid molecules, polypeptides, or vectors according to the present invention, the individual shows observable and/or measurable reduction in or absence of one or more of the symptoms associated with the liver-related disease, particularly NAFLD, NASH or cirrhosis; reduced morbidity and mortality, and improvement in quality of life issues. The above parameters for assessing successful treatment and improvement in the disease are readily measurable by routine procedures familiar to physician or authorized personnel.

“Preventing” refers to keeping from happening, and/or lowering the chance of the onset of, or at least one adverse effect or symptom of, a liver-related disease, particularly NAFLD, NASH or cirrhosis, disorder or condition associated with a deficiency in or absence of an organ, tissue or cell function.

“Individual” refers to an animal, preferably a mammal, more preferably a human. In one embodiment, the individual is a male. In another embodiment, the individual is a female. In one embodiment, an individual may be a “patient”, i.e. a warmblooded animal, more preferably a human, who/which is awaiting the receipt of, or is receiving medical care or was/is/will be the object of a medical procedure, or is monitored for the development of a liver-related disease, particularly NAFLD, NASH or cirrhosis. In one embodiment, the individual is an adult (for example a subject above the age of 18). In another embodiment, the individual is a child (for example a subject below the age of 18).

### BRIEF DESCRIPTION OF THE DRAWINGS

FIGS. 1A-1B is a set of photographs and graphs showing stable transfected IHH cells overexpressing SIRT6 allele variants. (A) Representative immunofluorescence images of Katushka2S staining showing the occurred lentivirus transfection for the empty vector and all of three SIRT6 variants (WT, N308K, N308K/A313S) in IHH cells. (B) The histogram shows quantification of the protein expression ratio SIRT6/GAPDH, H3K56Ac/Histone 3, H3K9Ac/Histone 3 relative to control group in IHH transfected cells (N=5-7). Data are presented as mean±SEM. *p<0.05 vs Empty group.

FIG. 2 is a graph showing that SIRT6 overexpression did not affect pAKT/AKT ratio in IHH. The histogram shows quantification of the protein expression ratio pAKT(ser473)/AKT relative to control group with or without insulin treatment of 100 nM for 30 minutes. Results revealed an increased protein levels in all groups after insulin stimulation, but with no differences among the groups themselves (N=6). Data are presented as mean±SEM.

FIGS. 3A-3B are scatter plots showing a metabolite profiling. (A) Score scatter plot of the PCA model of the human hepatocyte samples. First and second components are depicted. Model diagnostics (A=3; R2X=0.677; Q2X=0.278). The ellipse represents 95% confidence interval according to Hotelling's T2 test. (B) Score scatter plot of the PCA model of culture media extracts. Model diagnostics (A=3; R2X=0.662; Q2X=0.115). The ellipse represents 95% confidence interval according to Hotelling's T2 test.

FIGS. 4A-4B are heatmap representations showing amino acids profiling. (A) Heatmap representation of the changes in amino acids and derivatives for the comparisons between groups of human hepatocytes. The color code represents the log 2(fold-change). Student's t-test p-values: *p<0.05, **p<0.01; ***p<0.001. (B) Heatmap representation of the changes in amino acids and derivatives for the comparisons between culture media groups. The color code represents the log 2(fold-change). Student's t-test p values: *p<0.05. **p<0.01; ***p<0.001.

FIGS. 5A-5I are graphs showing the lipid profiling. (A) Heatmap representation of the changes in saturated (SFA), monounsaturated (MUFA) and polyunsaturated fatty acids (PUFA) for the comparisons between IHH lines. The color code represents the log 2(fold-change). (B) Boxplots of 18:1n-9 in IHH. (C) Boxplots of 20:3n-9 in hepatocytes. (D-I) Boxplots of PE(0:0/18:2) (D), PE(0:0/22:6) (E), PE(18:1/0:0) (F), PE(O-16:0/0:0) (G), PE(0:0/15:0) (H) and PE(0:0/16:1) (1) in IHH. Student's t-test p-values: not significant (ns), *p<0.05, **p<0.01; ***p<0.001.

FIGS. 6A-6C are graphs showing SIRT6 overexpression lowered basal collagen levels in IHH/LX2 spheroids. (A) The histogram shows quantification of percentage of collagen content in the spheroids structure. Collagen levels are significantly decreased in N308K/A313S group compared to Empty, WT and NK308K groups. (B) Quantification of soluble collagen content in the condition media of the different groups. All the group overexpressing one of the SIRT6 variant showed a significant decrease of about 30% in soluble collagen levels compared to Empty group. (C) mRNA levels of αSMA, COL1A1, TIMP1, Vimentin, MMP2 of the five spheroids groups. Data are presented as mean±SEM. *p<0.05 vs Empty group. **p<0.01 vs Empty group, § p<0.05 vs WT and N308K groups, #p<0.05 vs all other groups.

FIG. 7 is a graph showing the amino acids profiling in hepatocytes. Heatmap representing binary comparisons between hepatocyte groups per metabolite. Heatmap color codes for log 2 (fold-change) and Student's t-test p-values are indicated at the bottom of the figure.

FIG. 8 is a graph showing the amino acids profiling in the culture media. Heatmap representing binary comparisons between culture media groups per metabolite. Heatmap color codes for log 2 (fold-change) and Student's t-test p-values are indicated at the bottom of the figure.

FIGS. 9A-9F are graphs showing the amino acids profiling in hepatocytes. Boxplots of A) threonine, B) asparagine, C) aspartic acid, D) proline, E) arginine and F) citrulline levels in the hepatocytes. Student's t-test p-value: ns, p>0.05; *, p<0.05; **, p<0.01; ***p<0.001.

FIGS. 10A-10J are graphs showing the amino acids profiling in culture media. Boxplots of A) glutamic acid, B) asparagine, C) aspartic acid, D) arginine, E) cystine, F) serine, G) cystathionine, H) aminoadipic acid, I) citrulline and J) S-sulfocysteine in culture media groups. Student's t-test p-value: ns, p>0.05; *, p<0.05; **, p<0.01; ***p<0.001; ****p<0.0001.

FIGS. 11A-11C are graphs showing lipid profiling. (A) Heatmap representation of the influence of the number of carbons and double bonds in changes of diglycerides for comparisons between WT and empty vector transfected hepatocyte groups. (B) Heatmap representation of the influence of the number of carbons and double bonds in changes of triglycerides for comparisons between WT and empty vector transfected hepatocyte groups. (A-B) Color code represents the log 2(fold-change); the x axe denotes the number of carbons and the y axe denotes the number of double bonds. (C) Boxplots of TG (58:2) and TG (60:3) in IHH. Student's t-test p-value: ns, p>0.05; **, p<0.05; **, p<0.01.

FIGS. 12A-12B are graphs showing lipid profiling in IHH. Boxplots of SM (32:1) (A) and SM (42:3) (B) in IHH. Student's t-test p-value: ns, p>0.05; *, p<0.05; **, p<0.01.

FIGS. 13A-13D are graphs showing lipid profiling in hepatocytes. Boxplots of Cer(d18:1/20:0) (A), Cer(d18:1/21:0) (B), Cer(d18:1/22:0) (C) and Cer(d18:1/18:0) (D) in hepatocytes. Student's t-test p-value: ns, p>0.05; *, p<0.05; **, p<0.01.

FIGS. 14A-14C are histograms showing quantification of mRNA and protein expression upon transient expression by of SIRT6 and SIRTcent by AAV in LX-2 cells (stellate cells) using the target construct for clinical use. FIG. 14A-14B show RNA expression. FIG. 14C shows protein expression.

FIGS. 15A-15E are histograms and photographs showing quantification of mRNA and protein expression upon transient expression of SIRT6 and SIRTcent in organoids (stellate/hepatocyte cells) using the target construct for clinical use. FIG. 15A show RNA expression in spheroids. FIG. 15B-15C show basal conditions. FIG. 15D-15E shows fibrotic conditions.

FIGS. 16A-16B are histograms showing mRNA expression of several genes in primary hepatic stellate cells, in health of NASH conditions, with expression, or not, of SIRT6 wt or SIRT6 cent.

FIG. 17 is a histogram showing RNA expression of genes relates to lipid metabolism in IHH cells with expression, or not, of SIRT6 wt or SIRT6 cent.

FIGS. 18A-18D show different pattern of genes expression in tested groups (CTL (AAV-Luc), AAV-SIRT6-WT and AAV-SIRT6-Cent. 56 genes are differentially expressed in AAV-SIRT6-WT and AAV-SIRT6-Cent treated cells.

FIG. 19 represents the analysis of pathways differentially expressed between AAV-SIRT6-WT and AAV-SIRT6-Cent treated cells.

FIGS. 20A-20E are histograms showing posttranslational modifications (PTM) of histones by SIRT6 WT and SIRT6cent in 3T3-L1 adipocytes. FIG. 20A relates to HISTONE H4 G4KGGKGLGKGGAKR17. FIG. 20B relates to HISTONE H3.1/H3.3 K18QLATKAAR26. FIG. 20C relates to HISTONE H3.1/H3.3 K9STGGKAPR17. FIG. 20D relates to HISTONE H3.1 K27SAPATGGVKKPHR40. FIG. 20E relates to HISTONE H3.3 K27SAPSTGGVKKPHR40.

FIGS. 21A-21I are graphs and histograms showing the results in the in vivo model HF/DEN. FIG. 21A-21F show mice weight and weight gain. FIG. 21G-21I show mice organ weight.

FIGS. 22A-22D are histograms showing haematological examination in the HF/DEN mice model.

FIGS. 23A-23D are histograms showing protein expression levels of SIRT6 and B-catenin in the HF/DEN mice model.

FIGS. 24A-24D are graphs and histograms showing biodistribution in the HF/DEN mice model. FIG. 24A-24B show reporter (LUC) biodistribution, while FIG. 24C-24D show STRT6c biodistribution.

### DETAILED DESCRIPTION

This present invention relates to an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1 for use in the prevention and/or the treatment of non-alcoholic fatty liver disease (NAFLD), non-alcoholic steatohepatitis (NASH) or cirrhosis.

As used herein the expression “at least 75% identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% identity.

The level of identity of 2 polypeptides may be performed by using any one of the known algorithms available from the state of the art. Illustratively, the amino acid identity percentage may be determined using the CLUSTAL W software (version 1.83), the parameters being set as follows:
for slow/accurate alignments: (1) Gap Open Penalty: 10.00; (2) Gap Extension Penalty:0.1; (3) Protein weight matrix: BLOSUM;
for fast/approximate alignments: (4) Gap penalty: 3; (5) K-tuple (word) size: 1; (6) No. of top diagonals: 5; (7) Window size: 5; (8) Scoring Method: PERCENT.

Within the scope of the invention the sequence SEQ ID NO: 1 refers to the 361 amino acid residues sequence of wild type SIRT6 polypeptide. In practice, substitutions N308K and A313S refer to the mutations of the codon encoding the naturally occurring Asn (N) amino acid residue at position 308 in the SIRT6 polypeptide, and the Ser (S) amino acid residue at position 313 in the SIRT6 polypeptide, respectively.

Within the scope of the invention the sequence SEQ ID NO: 21 refers to the 1,068 nucleotides (bp) sequence of wild type SIRT6 polypeptide.

In some embodiments, the naturally occurring Asn (N) amino acid residue at position 308 in the SIRT6 polypeptide is encoded by codon “aac” at positions 922 to 924 of SEQ ID NO: 21. In some embodiments, the naturally occurring Ser (S) amino acid residue at position 313 in the SIRT6 polypeptide is encoded by codon “gcc” at positions 937 to 939 of SEQ ID NO: 21.

In certain embodiments, the N308K substitution is represented by a mutation of codon “aac” at positions 922 to 924 of SEQ ID NO: 21 into codon “aag” or codon “aaa”, preferably into codon “aag”. In other words, the N308K substitution is represented by a mutation of nucleotide “c” at positions 924 of SEQ ID NO: 21 into nucleotide “g” or nucleotide “a”, preferably into nucleotide “g”.

In certain embodiments, the A313S substitution is represented by a mutation of codon “gcc” at positions 937 to 939 of SEQ ID NO: 21 into a codon selected in a group consisting of codons “tcc”, “tct”, “tca” and “tcg”, preferably codon “tcc”. In other words, the A313S substitution is represented by one or two mutation(s) selected in a group consisting of a mutation of nucleotide “g” at positions 937 of SEQ ID NO: 21 into nucleotide “t”; a mutation of nucleotide “g” at positions 937 of SEQ ID NO: 21 into nucleotide “t” and of nucleotide “c” at positions 939 of SEQ ID NO: 21 into nucleotide “t”; a mutation of nucleotide “g” at positions 937 of SEQ ID NO: 21 into nucleotide “t” and of nucleotide “c” at positions 939 of SEQ ID NO: 21 into nucleotide “a”; and a mutation of nucleotide “g” at positions 937 of SEQ ID NO: 21 into nucleotide “t” and of nucleotide “c” at positions 939 of SEQ ID NO: 21 into nucleotide “g”.

In certain embodiments, the isolated polypeptide being a variant of SIRT6 has at least 75%, 80%, 85%, 90%, 95%, 96%, 97%, 98%, 99% or 100% identity with sequence SEQ ID NO: 6, SEQ ID NO: 7 or SEQ ID NO: 8. In some embodiments, the nucleic acid molecule is of sequence selected in the group comprising or consisting of SEQ ID NO: 6, SEQ ID NO: 7 and SEQ ID NO: 8.

As used herein, sequence SEQ ID NO: 6 refers to the nucleic acid sequence of the variant of SIRT6 with N308K substitution, in particular, with mutation of codon “aac” at positions 922 to 924 of SEQ ID NO: 21 into codon “aag”.

As used herein, sequence SEQ ID NO: 7 refers to the nucleic acid sequence of the variant of SIRT6 with A313S substitution, in particular, with mutation of codon “gcc” at positions 922 to 924 of SEQ ID NO: 21 into codon “tcc”.

As used herein, sequence SEQ ID NO: 8 refers to the nucleic acid sequence of the variant of SIRT6 with N308K and A313S substitutions, in particular, with mutation of codon “aac” at positions 922 to 924 of SEQ ID NO: 21 into codon “aag” and with mutation of codon “gcc” at positions 922 to 924 of SEQ ID NO: 21 into codon “tcc”.

In some embodiments, the variant of SIRT6 encoded by the isolated nucleic acid molecule as defined herein may have additional mutations compared to wild type SIRT6 polypeptide.

In some embodiments, the variant of SIRT6 encoded by the isolated nucleic acid molecule as defined herein has a deacylase and/or mono-ADP ribosyl transferase (mADPr) activity.

In practice, the deacylase activity and mono-ADP ribosyl transferase (mADPr) activity may be assayed accordingly to any suitable method from the state in the art, or a method adapted therefrom. Illustratively, deacylase activity may be assayed by contacting in vitro the variant of SIRT6 with histones, in the presence of NAD⁺, MgCl₂, DTT and performing a Western blot analysis using anti-H3K9ac and anti-H3K18ac antibodies. Illustratively, mono-ADP ribosyl transferase (mADPr) activity may be assayed by contacting in vitro the variant of SIRT6 with PARP1, in the presence of ZnCl₂, MgCl₂, NAD⁺, DTT, salmon sperm DNA and performing a Western blot analysis using anti-PADPR antibodies.

In certain embodiments, the variant of SIRT6 has at most about 90%, preferably at most about 50%, more preferably at most about 25% deacylase activity as compared to wild type SIRT6 (of sequence SEQ ID NO: 1). Within the scope of the invention, the expression “at most about 90%” encompasses about 90%, 85%, 80%, 75%, 70%, 65%, 60%, 55%, 50%, 45%, 40%, 35%, 30%, 25%, 20%, 15%, 10%, 5%, 1% or less.

In some embodiments, the variant of SIRT6 has at most least 100%, preferably at least about 200%, more preferably at least about 300% mono-ADP ribosyl transferase (mADPr) activity as compared to wild type SIRT6 (of sequence SEQ ID NO: 1). Within the scope of the invention, the expression “at least about 100%” encompasses about 100%, 120%, 140%, 160%, 180%, 200%, 220%, 240%, 260%, 280%, 300%, 350%, 400%, 450%, 500%, 550%, 600%, 650%, 700%, 750% or more.

In some embodiments, the nucleic acid molecule is a nucleic single or double stranded molecule. In some embodiments, the nucleic acid molecule is a DNA or RNA.

In one embodiment, the nucleic acid molecule is an RNA molecule. In some embodiments, the nucleic acid molecule is an RNA molecule selected from the list comprising messenger RNA (mRNA), transfer RNA (tRNA), ribosomal RNA (rRNA), micro-RNA (miRNA), small interfering RNA (siRNA), small nucleolar RNA (snoRNA), small nuclear RNA (snRNA), circular RNA (circ RNA), and long non-coding RNA (lncRNA). In a particular embodiment, the nucleic acid molecule is a mRNA.

In another embodiment, the nucleic acid molecule is a DNA molecule. In some embodiments, the nucleic acid molecule is a DNA molecule selected from the list comprising genomic DNA (gDNA) or complementary DNA (cDNA) molecule.

Non-Alcoholic Fatty Liver Disease (NAFLD) is characterized by an abnormal accumulation of triglycerides in the hepatocytes. It is related to metabolic syndrome, obesity, high caloric consumption and does not and does not usually include hepatic steatosis secondary to other causes.

NAFLD encompasses a broad spectrum of hepatic lesions in which two main entities can be distinguished: steatosis isolated or accompanied by minimal lobular inflammation (non-alcoholic fatty liver or NAFL) and non-alcoholic steatohepatitis (NASH).

In some embodiments, the isolated nucleic acid molecule according to the invention is for preventing and/or treating NAFLD. There are four stages of NAFLD: Stage 1 is simple fatty liver or steatosis, Stage 2 is non-alcoholic steatohepatitis (NASH), Stage 3 is fibrosis and Stage 4 is cirrhosis.

In some embodiments, the isolated nucleic acid molecule according to the invention is for preventing and/or treating Stage 1 or Stage 2 of NAFLD. In some embodiments, the isolated nucleic acid molecule according to the invention is for preventing and/or treating non fibrotic NAFLD. In some embodiments, the isolated nucleic acid molecule according to the invention is for preventing and/or treating non fibrotic stage of NAFLD.

In some embodiments, the isolated nucleic acid molecule according to the invention is for preventing and/or treating NASH (i.e., Stage 2 of NAFLD).

NASH is characterized by fat accumulation of (steatosis), hepatocytes injury due to fat accumulation, apoptosis and inflammation, fibrosis. There are 3 main stages of NASH. Stage 1 (mild) where fat in the liver exceeds 5% (steatosis), inflammation occurs, and the liver is bigger than normal. Typically in Stage 1, the liver will continue to function as it normally would, but may be compromised. This is also referred to as Compensated Cirrhosis, or NASH without Fibrosis. Stage 2 (moderate) where, in addition to stage 1 characteristics, scarring (fibrosis) begins to appear. Fibrosis can be classified as F1 through F4; Stage 2 of NASH involves fibrosis from F1 to F3. When a patient reaches this stage, the liver begins to deteriorate into liver failure. This is also referred to as NASH with Fibrosis. Stage 3 (severe) is the most severe stage of NASH, the disease deteriorates into full-on cirrhosis or liver cancer. When this happens, the only option left is a liver transplant.

In some embodiments, the isolated acid nucleic molecule, isolated polypeptide, vector, suspension, cell or pharmaceutical composition, is for preventing and/or treating Stage 1, Stage 2 or Stage 3 of NASH. In some embodiments, the isolated acid nucleic molecule, isolated polypeptide, vector, suspension, cell or pharmaceutical composition, is for preventing and/or treating Stage 1 or Stage 2 of NASH. In some embodiments, the isolated acid nucleic molecule, isolated polypeptide, vector, suspension, cell or pharmaceutical composition, is for preventing and/or treating Stage 1 of NASH. In some embodiments, the isolated acid nucleic molecule, isolated polypeptide, vector, suspension, cell or pharmaceutical composition, is for preventing and/or treating Stage 2 of NASH. In some embodiments, the isolated acid nucleic molecule, isolated polypeptide, vector, suspension, cell or pharmaceutical composition, is for preventing and/or treating cirrhosis.

In some embodiments, the isolated acid nucleic molecule, isolated polypeptide, vector, suspension, cell or pharmaceutical composition, is for preventing and/or treating Stage 1 or Stage 2 of NAFLD and Stage 1 or Stage 2 of NASH.

The present invention relates to an isolated polypeptide encoded by a nucleic acid molecule for use in the prevention and/or the treatment of NAFLD.

This invention relates to an isolated polypeptide being a variant of SIRT6 having at least 75% identity with sequence SEQ ID NO: 1, the variant having at least one mutation selected in the group comprising or consisting of a substitution N308K and a substitution A313S with respect to sequence SEQ ID NO: 1 for the prevention and/or treatment of NAFLD.

As used herein the expression “at least 75% identity” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% identity.

In certain embodiments, the isolated polypeptide being a variant of SIRT6 has at least 75%, 80%, 85%, 90%, 95% identity with sequence SEQ ID NO: 1, the variant having at least one mutation selected in the group comprising or consisting of a N308K substitution and an A313S substitution with respect to sequence SEQ ID NO: 1.

In certain embodiments, the isolated polypeptide being a variant of SIRT6 has at least 75%, 80%, 85%, 90%, 95%, 96%, 97%, 98%, 99% or 100% identity with sequence SEQ ID NO: 2, SEQ ID NO: 3 or SEQ ID NO: 4. In some embodiments, the polypeptide is of sequence selected in the group comprising or consisting of SEQ ID NO: 2, SEQ ID NO: 3 and SEQ ID NO:4.

As used herein, the sequence SEQ ID NO: 2 refers to the amino acid sequence the variant of SIRT6 with N308K substitution. In some embodiments, the polypeptide of sequence SEQ ID NO: 2 is encoded by a nucleic acid molecule of sequence SEQ ID NO: 6.

As used herein, the sequence SEQ ID NO: 3 refers to the amino acid sequence the variant of SIRT6 with A313S substitution. In some embodiments, the polypeptide of sequence SEQ ID NO: 2 is encoded by a nucleic acid molecule of sequence SEQ ID NO: 7.

As used herein, the sequence SEQ ID NO: 4 refers to the amino acid sequence of the variant of SIRT6 with N308K and A313S substitutions. In some embodiments, the polypeptide of sequence SEQ ID NO: 4 is encoded by a nucleic acid molecule of sequence SEQ ID NO: 8. In certain embodiments, the polypeptide is a recombinant polypeptide.

As used herein, the term “recombinant polypeptide” refers to a polypeptide encoded by an engineered nucleic acid and synthesized upon transformation of said engineered nucleic acid into a microorganism or transfection in an eukaryotic cell for synthesis purposes.

The present invention further relates to a vector comprising the isolated nucleic acid molecule as described herein before for use in the prevention and/or the treatment of NAFLD.

In some embodiment the vector comprising the isolated nucleic acid molecule is selected from the list comprising or consisting of minicircle nucleic acid, plasmids, cosmids, bacteriophages, bacterial artificial chromosome, viral vectors, linear DNA, enzymatic DNA, and Doggybone DNA.

In some embodiment the vector comprising the isolated nucleic acid molecule is selected from the list comprising minicircle nucleic acid, plasmids, cosmids, bacteriophages, or a bacterial artificial chromosome or viral vectors.

As used herein, the term “minicircle nucleic acid” encompasses non-viral vectors that merely comprise a gene expression cassette and are free of viral and/or bacterial backbone DNA elements from standard plasmids.

As used herein, the term “plasmid” is intended to refer to a small extra-genomic DNA molecule, most commonly found as circular double stranded DNA molecules that may be used as a cloning vector in molecular biology, to make and/or modify copies of DNA fragments up to about 15 kb (i.e., 15,000 base pairs). Plasmids may also be used as expression vectors to produce large amounts of proteins of interest encoded by a nucleic acid sequence found in the plasmid downstream of a promoter sequence.

A used herein, the term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid. Cosmids are typically used as cloning vector for DNA fragments ranging in size from about 32 to 52 kb.

As used herein, the term “bacterial artificial chromosome” or “BAC” refers to extra-genomic nucleic acid molecule based on a functional fertility plasmid that allows the even partition of said extra-genomic DNA molecules after division of the bacterial cell. BACs are typically used as cloning vector for DNA fragment ranging in size from about 150 to 350 kb.

As used herein, the term “enzymatic DNA” refers to a synthetic, linear, double-stranded, closed-ended DNA molecule. As used herein, the term “Doggybone DNA” refers to a minimal, linear, double stranded and covalently closed DNA construct.

In practice, the vector comprising the nucleic acid molecule encoding a variant of SIRT6 may be in the form of a plasmid, in particular resulting from the cloning of a nucleic acid of interest into a nucleic acid vector. In some embodiments, non-limitative suitable nucleic acid vectors are pBluescript vectors, pET vectors, pETduet vectors, pGBM vectors, pBAD vectors, pUC vectors. In one embodiment, the plasmid is a low copy plasmid. In one embodiment, the plasmid is a high copy plasmid.

In some embodiments, the vector is a viral vector. In some embodiments, the viral vector is selected in a group comprising or consisting of an adenovirus; an adeno-associated virus (AAV); an exosome-associated AAV (exo-AAV); an exosome; an alphavirus; a herpesvirus; a retrovirus, such as, e.g., a lentivirus or a non-integrative lentivirus; vaccinia virus; a baculovirus; or virus like particles such as, e.g., particles derived from Hepatitis B virus, Parvoviridae, Retroviridae, Flaviviridae, Paramyxoviridae or bacteriophages. In one embodiment, the exosome comprises at least one DNA molecule, RNA molecule and/or protein, preferably the variant of SIRT6 according to the invention or a nucleic acid encoding thereof.

In some embodiments, the vector is a viral vector. In some embodiments, the viral vector is selected in a group comprising or consisting of an adenovirus; an adeno-associated virus (AAV); an exosome-associated AAV (exo-AAV); an alphavirus; a herpesvirus; a retrovirus, such as, e.g., a lentivirus or a non-integrative lentivirus; vaccinia virus; a baculovirus; or virus like particles such as, e.g., particles derived from Hepatitis B virus, Parvoviridae, Retroviridae, Flaviviridae, Paramyxoviridae or bacteriophages.

In some embodiments, the viral vector of the invention is selected from the group comprising or consisting of adeno-associated viral vector (AAV), exosome-associated AAV vector (exo-AAV), exosorne, adenoviral vector, retroviral vector, lentivirus and herpes virus vector.

In some embodiments, the viral vector of the invention is selected from the group comprising or consisting of adeno-associated viral vector (AAV), exosome-associated AAV vector (exo-AAV), adenoviral vector, retroviral vector, lentivirus and herpes virus vector.

In some embodiments, the viral vector of the invention is an adeno-associated viral vector (AAV), preferably an AAV serotype 2 or AAV serotype 5.

In certain embodiments, the vector, in particular the viral vector, is an exo-AAV vector. As used herein, exo-AAV refers to a vector wherein an adeno-associated virus (AAV) vector, or parts thereof, is associated with an extracellular vesicle (also referred to as exosome), wherein the AAV vector is partially fused, embedded or internalized in the extracellular vesicle. The extracellular vesicle may express specific proteins or markers, for example for targeting purposes.

In certain embodiments, the vector is an exosome, preferably an exosome comprising a payload or cargo, more preferably an exosome comprising a DNA molecule, RNA molecule and/or protein, even more preferably a variant of SIRT6 according to the invention or a nucleic acid encoding thereof.

In another embodiment, the viral vector is a retrovirus or lentivirus. In a preferred embodiment, the viral vector is a lentivirus

In certain embodiments, the vector, in particular the viral vector, does not cross the blood-brain barrier. In some alternative embodiments, the vector, in particular viral vector crosses the blood-brain barrier.

In some embodiments, the vector, in particular the viral vector, comprises a promoter sequence suitable for gene expression in mammalian individuals, preferably in human individuals.

Non-limitative examples of promoter sequence suitable for gene expression in mammalian individuals, preferably in human individuals, include CMV (human cytomegalovirus) promoter, EF1α (human elongation factor 1 alpha) promoter, SV40 (Simian vacuolating virus 40) promoter, PGK1 (phosphoglycerate kinase) promoter, UbC (human ubiquitin C) promoter, ColA2 promoter, Col1A1 promoter, Col3A1 promoter, and the like.

In certain embodiments, the promoter sequence is preferably the EF1α promoter.

In some embodiments, the vector, in particular the viral vector, further comprises a nucleic acid sequence that facilitates the nuclear localization of the polypeptide encoded by the nucleic acid molecule according to the invention into a target recipient cell. In practice, these nuclear localization signal (NLS) have been abundantly discussed in the state of the art.

The present invention further relates to a suspension comprising a vector for use in the prevention and/or the treatment of NAFLD.

In one aspect, the invention relates to a suspension comprising a vector according to the invention.

In some embodiments, the suspension further comprises a fluid including one or more ingredients selected from the group consisting of water, saline, phosphate buffered saline, dextrose, glycerol, ethanol and a combination thereof.

In certain embodiment, the suspension is formulated for intravenous infusion. In practice, the suspension formulated for intravenous infusion may comprise saline (e.g., 0.9% NaCl); lactated Ringers; 5% dextrose; a colloid, such as, e.g., albumin; the like; and any combination thereof

The present invention further relates to a cell expressing the polypeptide, the cell being preferably transfected with an isolated nucleic acid molecule, or a vector, for use in the prevention and/or the treatment of NAFLD.

In certain embodiments, the cell is a eukaryote cell, preferably an animal cells, more preferably a mammalian cell. As used herein “mammalian cell” includes nonhuman mammalian cells and human cells. In some embodiments, the cell is a human cell.

In some embodiments, the cell is selected in the group comprising or consisting of nerve cells, bone cells, breast cells, red blood cells, white blood cells, cartilage cells, epithelial cells, endothelial cells, skin cells, muscle cells, bladder cells, kidney cells, liver cells, prostate cells, cervix cells, ovarian cells, pulmonary cells, retinal cells, conjunctival cells, corneal cells, fat cells, and the like.

It is to be understood that the cell according to the invention has been transfected with an isolated nucleic acid molecule according to the invention, or transduced with an isolated nucleic acid molecule according to the invention, or contacted with the vector or the suspension containing the nucleic acid molecule according to the invention. Therefore, the cell contains the nucleic acid molecules either integrated or not in its genome. In practice, because the vector is an expression system, the nucleic acid molecule encoding the variant of SIRT6 is present within the cell in a form allowing its expression and its final location, i.e., the cell nucleus and cytoplasm.

The present invention further relates to a pharmaceutical composition comprising (i) an isolated nucleic acid molecule, or an isolated polypeptide, or a vector, and (ii) a pharmaceutically acceptable excipient, for use in the prevention and/or treatment of NAFLD.

In some embodiments, a suitable pharmaceutically acceptable carrier according to the invention includes any and all conventional solvents, dispersion media, fillers, solid carriers, aqueous solutions, coatings, antibacterial and antifungal agents, isotonic and absorption delaying agents, and the like. In certain embodiments, suitable pharmaceutically acceptable carriers may include, water, saline, phosphate buffered saline, dextrose, glycerol, ethanol and a mixture thereof. In some embodiments, pharmaceutically acceptable carriers may further comprise minor amounts of auxiliary substances such as wetting or emulsifying agents, preservatives or buffers, which enhance the shelf life or effectiveness of the cells. The preparation and use of pharmaceutically acceptable carriers are well known in the art.

In some embodiments, the nucleic acid molecule, the polypeptide, the vector, the suspension, or the pharmaceutical composition according to the invention is to be administered to an individual in need thereof by any suitable route, i.e., by a dermal administration, by an oral administration, a topical administration or a parenteral administration, e.g., by injection, including a sub-cutaneous administration, a venous administration, an arterial administration, in intra-muscular administration, an intraocular administration and an intra-auricular administration.

In certain embodiments, the nucleic acid molecule, the polypeptide, the vector, the suspension, or the pharmaceutical composition according to the invention is to be administered to an individual in need thereof by a dermal administration. In certain embodiments, the nucleic acid molecule, the polypeptide, the vector, the suspension, or the pharmaceutical composition according to the invention is associated with a composition enabling and/or facilitating dermal administration, e.g., by increasing dermal tropism or by increasing dermal barrier penetration. In some embodiments, the dermal administration enables a prolonged liberation of the nucleic acid molecule, the polypeptide, the vector, the suspension, or the pharmaceutical composition according to the invention.

In certain embodiments, the nucleic acid molecule, the polypeptide, the vector, the suspension, or the pharmaceutical composition according to the invention is to be administered to an individual in need thereof by an intravenous administration, in particular by intravenous infusion or intravenous injection.

Within the scope of the instant invention, the therapeutically effective amount of the nucleic acid molecule, the polypeptide, the vector, the suspension, or the pharmaceutical composition according to the invention, to be administered may be determined by a physician or an authorized person skilled in the art and can be suitably adapted within the time course of the treatment.

In certain embodiments, the therapeutically effective amount to be administered may depend upon a variety of parameters, including the material selected for administration, whether the administration is in single or multiple doses, and the individual's parameters including age, physical conditions, size, weight, gender, and the severity of the age-related disease to be treated.

In certain embodiments, a therapeutically effective amount of the isolated polypeptide, or the pharmaceutical composition comprising the isolated polypeptide according to the invention, agent may range from about 0.001 mg to about 3,000 mg, per dosage unit, preferably from about 0.05 mg to about 100 mg, per dosage unit.

Within the scope of the instant invention, the expression “from about 0.001 mg to about 3,000 mg” includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 10 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 15 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the isolated polypeptide or the pharmaceutical composition comprising the isolated polypeptide according to the invention, may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 25 mg/kg, from about 0.1 mg/kg to about 10 mg/kg, and more preferably from about 1 mg/kg to about 25 mg/kg, of subject body weight per day. Within the scope of the instant invention, the expression “from about 0.001 mg/kg to about 100 mg/kg” includes about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 mg/kg, 0.02 mg/kg, 0.03 mg/kg, 0.04 mg/kg, 30 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg and 100 mg/kg.

In some embodiments, the therapeutically efficient amount of the isolated nucleic acid molecule, vector, or pharmaceutical composition according to the invention is ranging from about 10¹ to about 10¹⁵ copies per ml. In practice, a therapeutically efficient amount includes about 10¹, 5×10¹, 10², 5×10², 10³, 5×10³, 10⁴, 5×10⁴, 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ copies per ml. In certain embodiments, the therapeutically efficient amount is from about 10¹ to about 10¹ copies per cm3, which includes about 10¹, 5×10¹, 10², 5×10², 10³, 5×10³, 10⁴, 5×10⁴, 10δ, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ per cm3. In some embodiments, the therapeutically efficient amount is from about 10¹ to about 10⁵ copies per dose, which includes about 10¹, 5×10¹, 10², 5×10², 10³, 5×10³, 10⁴, 5×10⁴, 10⁵, 5×10⁵, 10⁶, 5×10⁶, 10⁷, 5×10⁷, 10, 5×10⁸, 10⁹, 5×10⁹, 10¹⁰, 5×10¹⁰, 10¹¹, 5×10¹¹, 10¹², 5×10¹², 10¹³, 5×10¹³, 10¹⁴, 5×10¹⁴ and 10¹⁵ copies per dose.

The invention also relates to the use of an isolated nucleic acid molecule, or an isolated polypeptide, or a vector according to the instant invention, for the preparation or the manufacture of a medicament for the prevention and/or the treatment of NAFLD.

In some further aspect, the invention pertains to a method for the prevention and/or the treatment of NAFLD in an individual in need thereof, comprising the administration of a therapeutically efficient amount of an isolated nucleic acid molecule, or an isolated polypeptide, or a vector according to the instant invention.

In certain embodiments, the isolated nucleic acid molecule, isolated polypeptide, vector, suspension, or pharmaceutical composition according to the instant invention is to be co-administered, or sequentially administered, with a drug suitable for preventing and/or treating NAFLD, in particular Stage 1 of NAFLD and Stage 1 and Stage 2 of NASH.

As used herein, the term “co-administered” refers to a simultaneous administration of the active principles. As used herein, the term “sequentially administered” refers to an administration of a first active principle before or after the administration of a second active principle.

Another aspect of the invention relates to a kit comprising (i) an isolated nucleic acid, an isolated polypeptide molecule, a vector, or a suspension according to the instant invention, and (ii) means to administer the isolated nucleic acid molecule, the isolated polypeptide, the vector, or the suspension.

In some embodiments, the means to administer the isolated nucleic acid molecule, the isolated polypeptide, the vector, or the suspension include a syringe or a catheter.

In certain embodiments, the individual in need thereof is a mammalian individual, preferably a human individual.

In some embodiments, the individual is suffering or at risk of suffering NAFLD, in particular Stage 1 or Stage 2 of NAFLD, more particularly Stage 1 or Stage 2 of NASH.

In some embodiments, the variant of SIRT6 according to the invention downregulates the expression of one or more of the following genes: αSMA, TIMP1, TP63, COL1A1. In some embodiments, the variant of SIRT6 according to the invention downregulates the b-catenin pathway. In some embodiments, the variant of SIRT6 according to the invention downregulates the glucocorticoid pathway.

As used herein, “downregultates” means decreases or reduces by at least 1%, 5%, 10%, 50%, or more, the mRNA and/or protein expression of the gene in a cell and/or a tissue compared to an untreated cell or tissue.

In some embodiments, the variant of SIRT6 according to the invention upregulates the expression of one or more of the following genes: MMP2, FN1, LOXL2, PDGFRb, FABP5, SGMS1.

As used herein, “upregulates” means increases or enhances by at least 1%, 5%, 10%, 50%, or more, the mRNA and/or protein expression of the gene in a cell and/or a tissue compared to an untreated cell or tissue.

### EXAMPLES

The present invention is further illustrated by the following examples.

### Example 1: SIRT6 Variants Overexpression in Immortalized Human Hepatocytes (IHH)

### Material and Methods

### Cell Culture

Human hepatocytes cell line (IHH), isolated and immortalized by lentiviral transduction with the SV40T antigen and hTERT were maintained in phenol red-free Dulbecco's modified Eagle's medium (DMEM/F-12) containing 1×10⁻⁶ M dexamethasone, 1×10⁻¹² M human insulin (Humalog, Lilly) 10% FBS and 1% penicillin/streptomycin. The cell culture medium was changed every 2 days and the cells were subcultured using TrypLE Express when reaching 90% confluence.

### Immunoblotting Analyses

Briefly, cells were harvested from using TrypLE Express, washed with 1×PBS and centrifuged at 300 g. Supernatant was discarded and the obtained pellet was resuspended in 1×RIPA lysis buffer (20-188, Millipore, USA) supplemented with Halt™ Protease and Phosphatase Inhibitor Cocktail (100×, ThermoFisher) and lysed on ice (4° C.) for 30 minutes with vigorous vortexing every 10 min. The samples were then centrifuged at 10000 g for 10 min at 4° C., supernatant was transferred to new microfuge tube and concentration of protein was measured by Pierce™ BCA Protein Assay Kit (23225, ThermoFisher) according to manufacturer's instruction. Equal amount of protein samples (at least 20 g) was mixed with 1× Laemmli Sample buffer (1610747, 4×, Bio-Rad) and after heating at 95° C. for 5 min and cooling on ice, equal volume of proteins (40 μl) were loaded on 10% Mini-PROTEAN® TGX Stain-Free™ Protein Gels (4568034, Bio-Rad) and separated by electrophoresis running at 120 volts for 45 minutes. Protein transfer was performed on PVDF membranes using Trans-Blot Turbo RTA Mini 0.45 μm LF PVDF Transfer Kit (1704274, Bio-Rad) and Bio-Rad Trans-Blot Turbo Transfer System at 1.3 A and 25V for 10 min. Membranes were then blocked with 5% bovine serum albumin (BSA, P6154, BioWest) dissolved in TBST buffer (20 mM Tris-HCl, pH 7.6, 140 mM NaCl, 0.1% Tween 20) for at least 30 minutes and incubated with the specific primary antibodies (see below) diluted in TBST blocking solution, at appropriate dilutions. Following three washes in TBST buffer, membranes were incubated with secondary antibodies conjugated with horseradish peroxidase diluted in TBST blocking buffer. After three further washes with TBST, protein levels were detected by Clarity Western ECL Substrate (1705061, Bio-Rad) and the signal detected on Bio-Rad ChemiDoc XRS+ imaging systems. For quantitative measurement, the scanned membranes were analyzed using the Image Lab™ Software (Bio-Rad).

The following antibodies were used: Cell Signaling Technology (MA, USA)—rabbit anti-Akt (1:1000), rabbit anti-Phospho-Akt (Ser473) (1:1000), rabbit anti Histone H3 (DIH2, 1:1000), Abeam (UK)—rabbit anti Collagen I (1:1000), rabbit anti SIRT6 antibody (1:1000, EPR18463), ThermoFischer Scientific (CA, USA)—mouse IgG1 GAPDH monoclonal HRP conjugated antibody (1:2000), secondary goat Anti-rabbit IgG HRP-linked (1:2000) and secondary goat Anti-mouse IgG HRP-linked (1:2000).

### Results

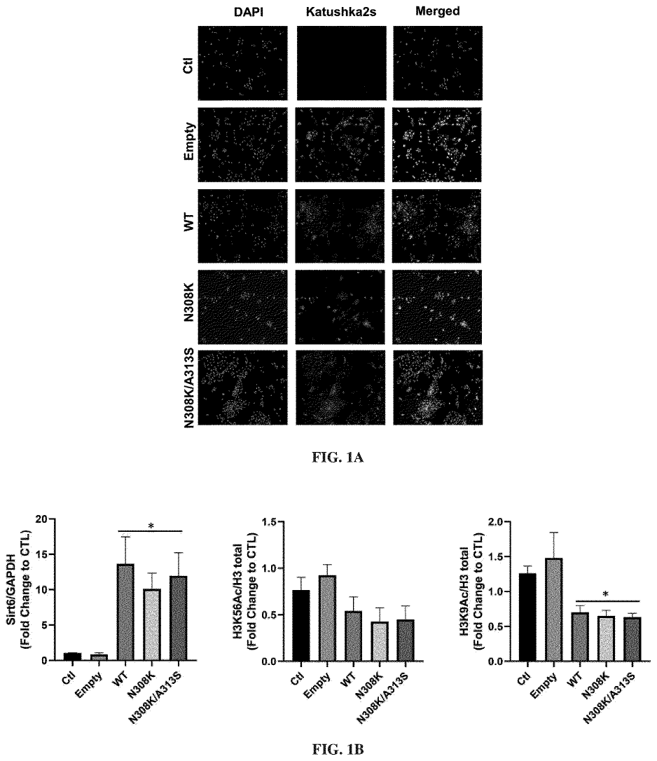

FIG. 1A shows the signal of the far-red fluorescence protein Katushka2S contained in the LV cassette, alone in the empty group, or together with one of SIRT6 versions (WT, N308K or N308K/A313S), demonstrating the successful infection. No Katushka2S signal was detected in the IHH control cells. SIRT6 protein expression was measured by Western Blot (FIG. 1B), confirming the strong increase in SIRT6 levels in the groups transfected with LV-SIRT6 compared to either empty or CTL cells. SIRT6 is actively recruited to target gene promoters and represses gene transcription by removing acetylation of H3K9 and H3K56 sites. Accordingly, in the SIRT6 overexpression (OE) groups the levels of acetylated histone H3K56 were significantly reduced, while H3K9Ac showed a decreased trend (FIG. 1B), supporting the fact that, along with the increased SIRT6 expression, there was a concomitant increase in its deacetylase activity.

### Example 2: Overexpression of Centenarian Variants of SIRT6 Dramatically Alters the Metabolomic Profiles of IHH, without Changes in Insulin Sensitivity

### Material and Methods

### Immunoblotting Analyses

See Example 1.

### Metabolomics

Metabolic profiling was performed by mass spectrometry coupled to ultra-high-performance liquid chromatography (UHPLC-MS). Cell pellets or cell culture media were resuspended/diluted in cold extraction solvents spiked with metabolites not detected in the unspiked cell extracts (internal standards) and incubated at −20° C. for 1 h. The samples were then vortexed and centrifuged at 18,000×g at 4° C. for 5 min, and the supernatants were collected and incubated at 4° C. while the cell pellets were again resuspended in cold extraction solvents and incubated for a further 1 h at −20° C. The samples were again vortexed and centrifuged at 18,000×g at 4° C. for 5 min and the supernatants were collected and pooled with the previous supernatant samples. The supernatants were then dried under vacuum, reconstituted in water and resuspended with agitation for 15 min before being centrifuged at 18,000×g for 5 min at 4° C. and transferred to vials for UHPLC-MS analysis. Two different types of quality control (QC) samples were used to assess the data quality: (i) a QC calibration sample to correct the different response factors between and within batches and (ii) a QC validation sample to assess how well the data pre-processing procedure improved data quality. Randomized sample injections were performed, with each of the QC calibration and validation extracts uniformly interspersed throughout the entire batch run. A specific UHPLC-MS method was used.

Data normalization and quality control: Normalization factors were calculated for each metabolite by dividing their intensities in each sample by the recorded intensity of an appropriate internal standard in that same sample. The most appropriate internal standard for each variable was defined as that which resulted in a minimum relative standard deviation after correction, as calculated from the QC calibration samples over all the analysis batches. In general, best internal standard trends followed chemical structural similarities between spiked compounds and endogenous variables. Robust linear regression (internal standard corrected response as a function of sample injection order) was used to estimate in the QC calibration samples any intra-batch drift not corrected for by internal standard correction. For all variables, internal standard corrected response in each batch was divided by its corresponding intrabatch drift trend, such that normalized abundance values of the study samples were expressed with respect to the batch averaged QC calibration serum samples (arbitrarily set to 1). Any remaining sample injection variable response zero values in the corrected dataset were replaced with missing values before generating the final dataset that was used for study sample statistical analyses.

Univariate data analysis: univariate statistical analyses were also performed for each metabolite measured in the hepatocytes and culture medium samples, calculating group percentage changes and Student's t-test p-value (or Welch's t test where unequal variances were found) for the comparisons among groups: WT vs. Empty; N308K vs. Empty; N308K/A313S vs. Empty; N308K vs. WT; N308K/A313S vs. WT; and N308K/A313S vs. N308K. In order to help in the visualization of the results, a heatmap per type of sample was generated displaying the results of the comparisons mentioned above. These heatmaps display the log 2 (fold-change) of the metabolites included in the analysis together with the Student's t-test for the comparisons performed. For each metabolite, changes between subgroups were calculated as the base 2 logarithm of fold-change. Darker blue and red colors indicate higher drops and elevations of the metabolite levels, respectively. These values are accompanied by a significance level based on p-values from Student's t-test. Three levels of increasing significance are considered: p<0.05, p<0.01 and p<0.001.

### Results

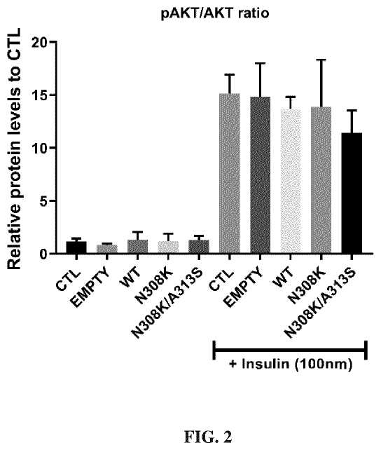

Because alterations of insulin receptor substrate PI3K (phosphoinositide 3-kinase) and AKT signaling pathways are well known to be closely associated with metabolic disorders, liver steatosis and insulin resistance the level of insulin sensitivity/activation PI3K/AKT pathway were assessed by immunoblotting upon overexpression of SIRT6 and its longevity variants in IHH cells. After overnight serum and glucose starvation of IHH cells, overexpressing or not SIRT6 and its allelic variants, we stimulated them with human insulin solution (100 nM) for 30 minutes and then we measured the pAKT(Ser473) protein levels. We did not observe any significant difference of pAKT(Ser473) levels among the conditions, either with or without insulin administration (FIG. 2).

Further, a depth metabolic profiling in IHH cells and their supernatants was conducted upon overexpression of SIRT6 and its allelic variants. Ultra-high performance liquid chromatography-mass spectrometry (UHPLC-MS) platforms optimized for extensive coverage of the metabolome were used, allowing the optimal profiling of: (1) Fatty acyls, bile acids, steroids and lysoglycerophospholipids; (2) Glycerolipids, glycerophospholipids, sterol lipids and sphingolipids; (3) Amino acids and derivatives. A total of 296 and 282 metabolic features were detected in the analyzed cell pellets and the culture media samples, respectively.

### PCA Analysis

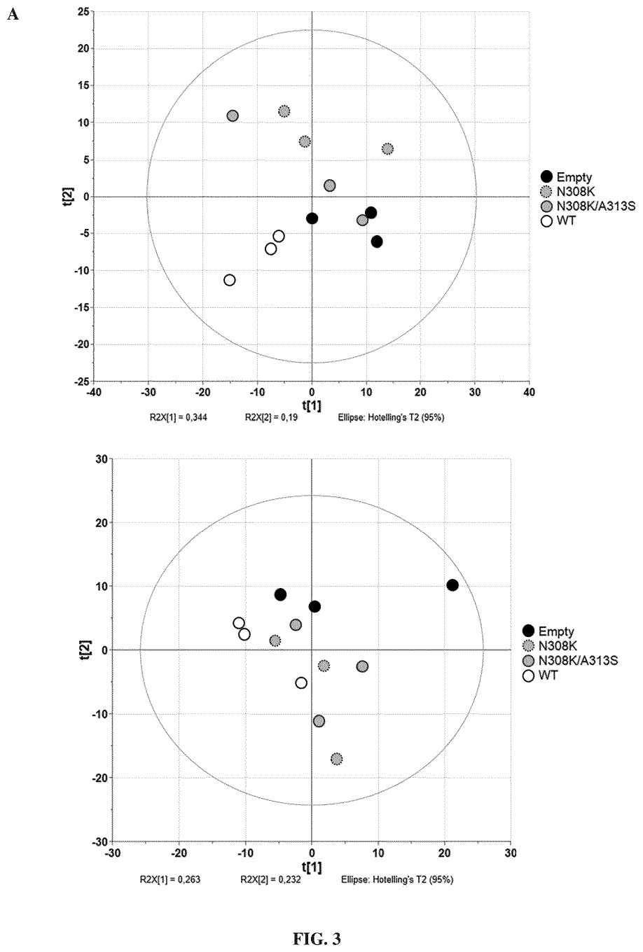

First, a principal component analysis (PCA) of hepatocyte extracts was performed for the four IHH cell lines: empty, WT, N308K or N308K/A313S. The score scatter plot of this PCA shows a separation of WT, N308K and N308K/A313S samples when the first t[1] and second t[2] components are depicted, with the WT being more segregated compared to the other groups (FIG. 3A). The t[1] and t[2] components explain the 34.4% and 19.0%, respectively, of the variability among samples. Similarly, a PCA analysis of IHH culture media samples was performed. However, the separation between groups (FIG. 3B) was not as clear as in the case of the hepatocyte extracts (FIG. 3A).

### General Metabolites Changes (Heat Maps)

A higher number of changes in the metabolites' levels were found in the analysis of human hepatocytes (FIG. 7) than in the comparisons performed between culture media groups (FIG. 8). Most of the changes were observed between the WT samples and the other groups, being a fewer number of metabolites altered between both mutant groups and when they were compared to the hepatocytes transfected with the empty vector (FIG. 7). It is remarkable the reduction of the levels of most glycerophospholipids in the cells transfected with the WT SIRT6 sequence when compared to the empty vector (FIG. 7), although this reduction was not observed in the culture media (FIG. 8).

It is also relevant that almost the complete profile of amino acids (AA) was increased in the mutant groups when compared to the hepatocytes transfected with the WT STRT6 sequence (FIG. 7). However, fewer changes were observed in the levels of amino acids in the case of the culture media (FIG. 8). Increments in several fatty acid (FA) and glycerophospholipids (especially lysophosphatidylethanolamines, LPE) were also detected for the comparisons between the hepatocytes transfected with the SIRT6 mutant sequences and the WT one, although a higher number of species were altered in the group N308K than in the N308K/A313S (FIG. 7). A reduction in ceramides was only detected in N308K group when compared to the WT group, but not for N308K/A313S group (FIG. 7). Several glycerophospholipids were also increased in the culture media of the mutant groups when compared to the WT group, especially ether-linked glycerophosphatidylcholines (ether-PC) in the comparison N308K vs. WT (FIG. 8).

### Amino Acids

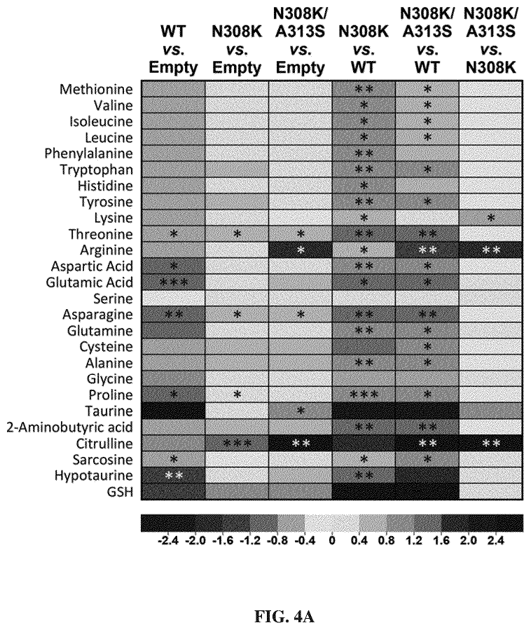

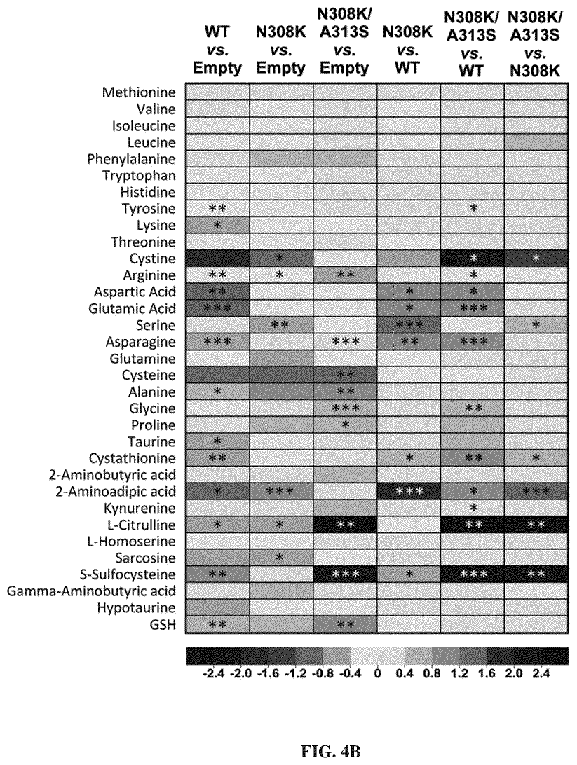

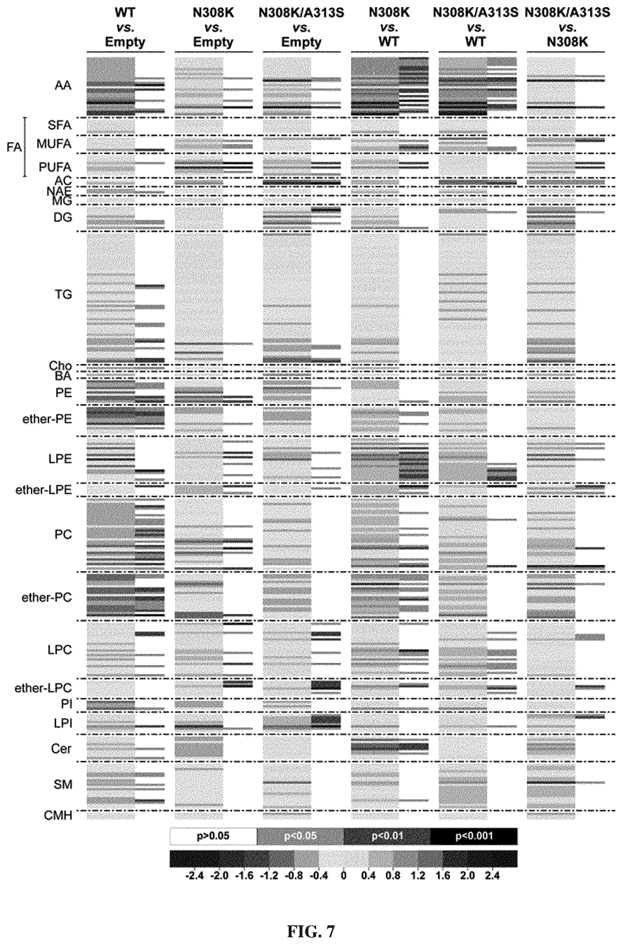

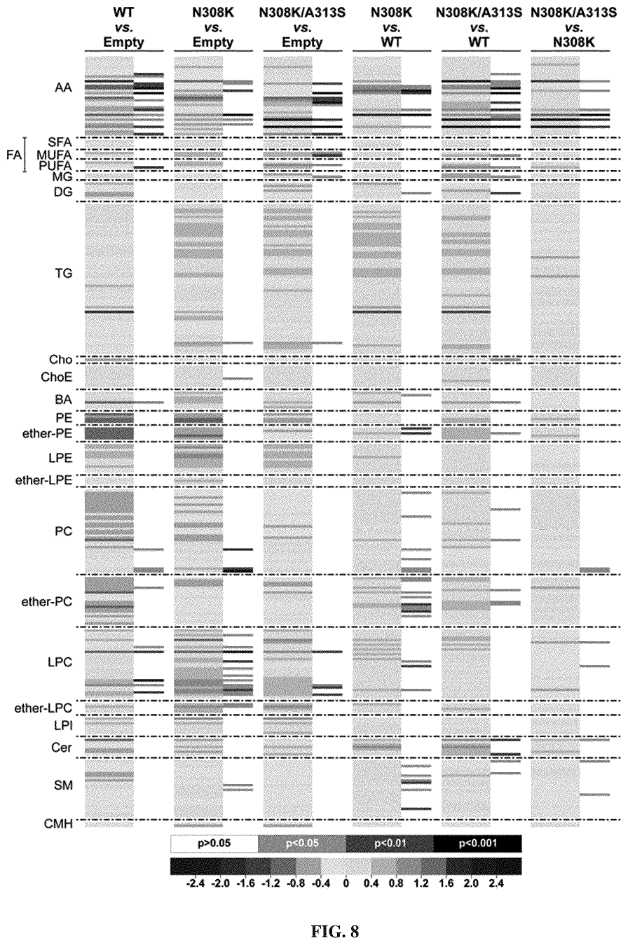

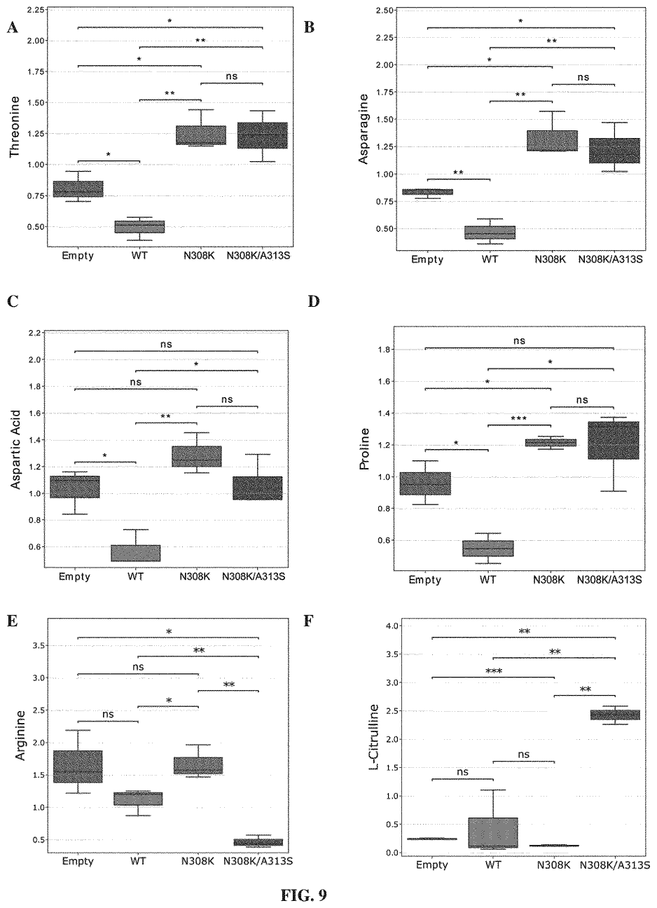

Levels of several amino acids were reduced in the hepatocytes transfected with the WT SIRT6 sequence when compared to the empty vector: threonine, aspartate, glutamate, asparagine, proline, sarcosine and hypotaurine (FIG. 4A). Stunningly, however, most of these metabolites were increased in the mutant groups when compared to the empty-vector group (FIG. 4A). Some examples of these changes are included in the boxplots of FIG. 9. Although it is remarkable that almost the complete profile of amino acids and derivatives was increased in the comparisons of mutant groups vs. the WT group (FIG. 4A), the only exception was the reduction in the levels of arginine in the N308K/A313S group when compared to the other groups (FIG. 9). In contrast, very few changes were detected between hepatocyte groups transfected with the mutations. Among them, a reduction of the levels of arginine and lysine and an increment of citrulline levels were found in the N308K/A313S group when compared to N308K group (FIG. 4 and FIG. 9).

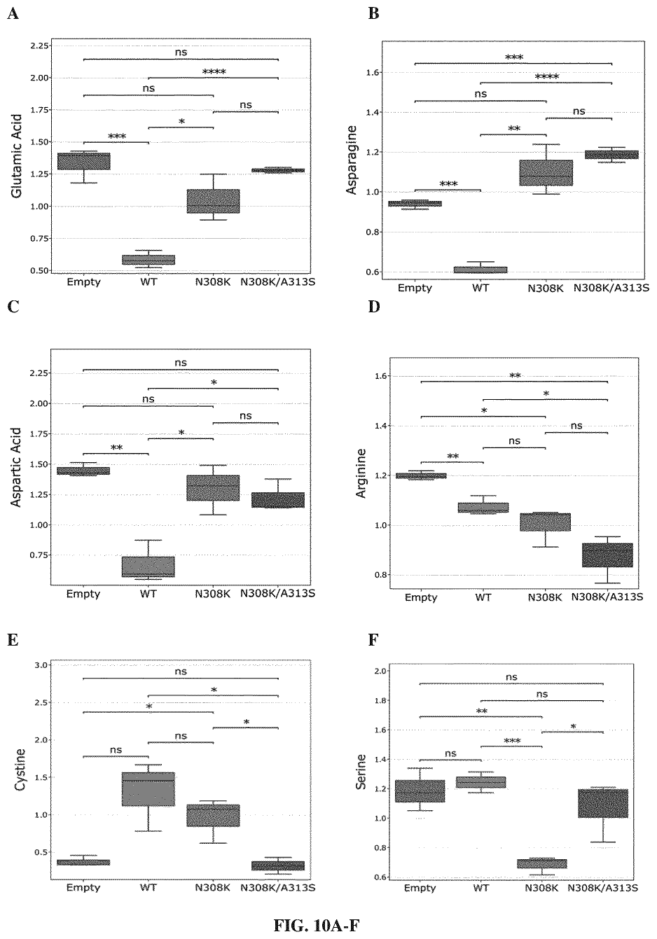

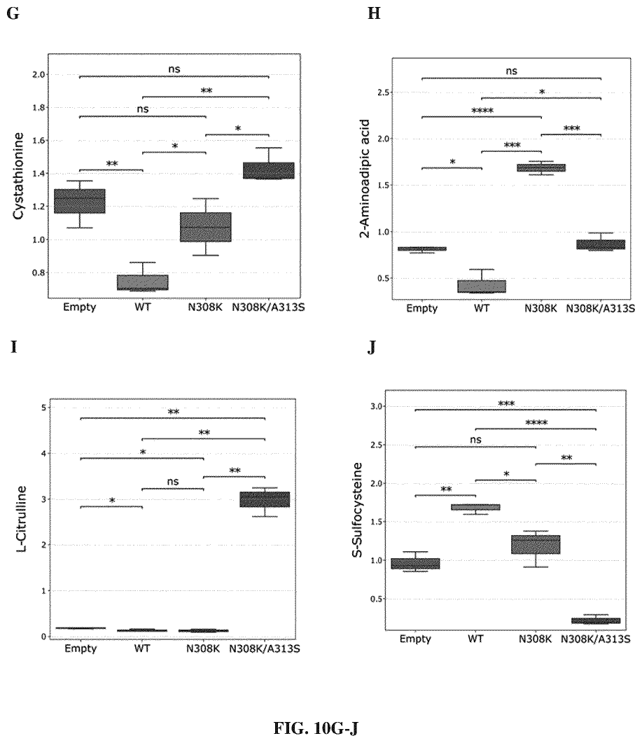

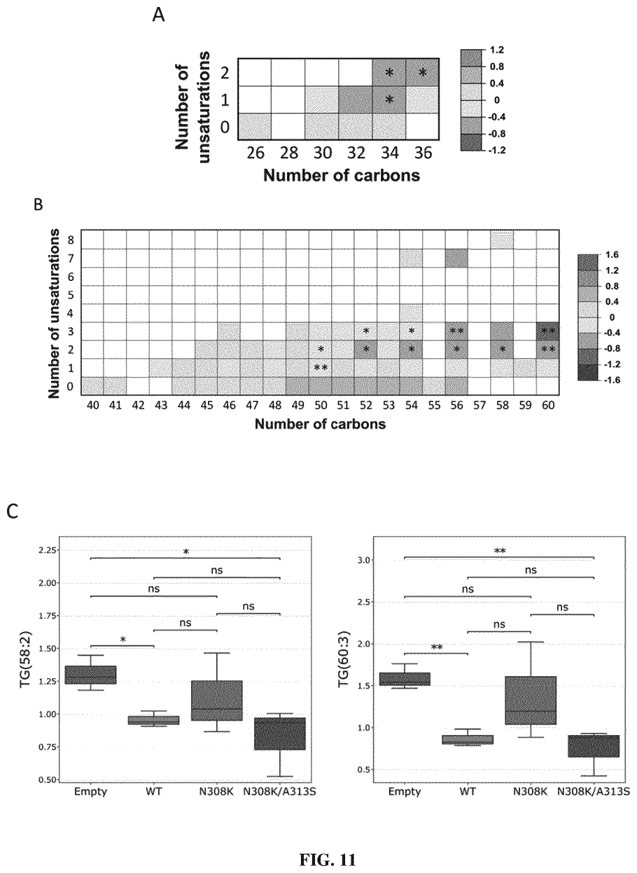

Changes in amino acids levels were also observed between culture media groups (FIG. 4B). The most significant changes (lower p-values) in the amino acid levels of WT-transfected hepatocytes' culture medium when compared to the empty vector group were the reduction of aspartate, glutamate, asparagine and arginine (FIG. 4A and FIG. 10). These reductions were also found in the cell pellets (FIG. 4A) except for arginine, for which the reduction did not reach a p-value <0.05 (FIG. 9). Levels of asparagine, aspartic acid or glutamic acid were increased in the culture media of mutant cells compared to WT samples (FIG. 10), but arginine was reduced in N308K/A313S samples when compared to WT group, as also detected in the hepatocytes (FIGS. 10 and 11, respectively). Levels of several amino acids were also altered between both mutant groups: cystine, serine, cystathionine, aminoadipic acid, citrulline and sulfocysteine (FIG. 10).

### Saturated Fatty Acids

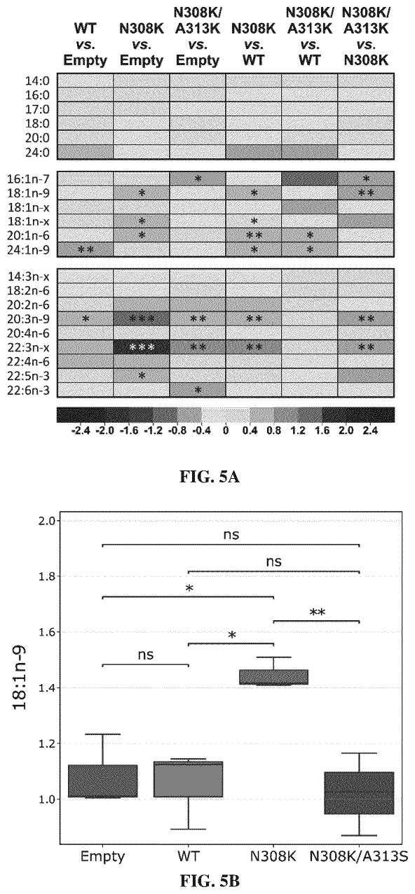

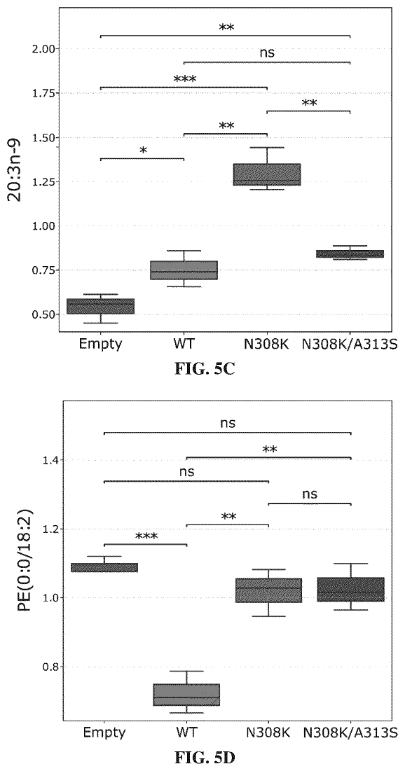

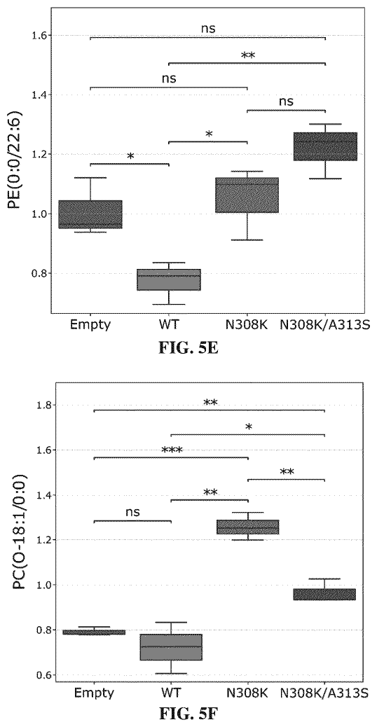

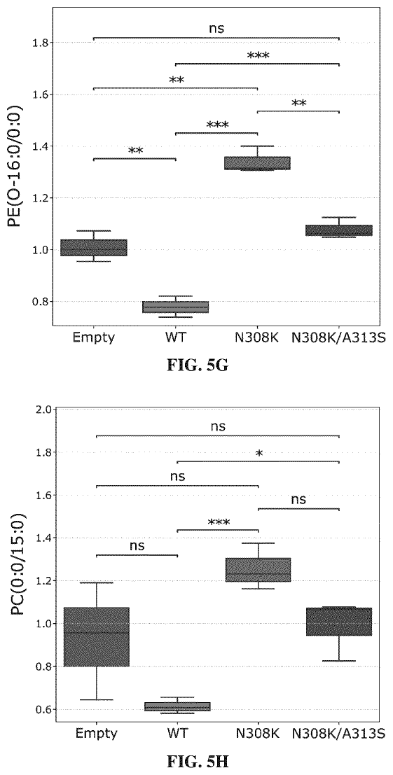

No changes were detected in the levels of saturated fatty acids (SFA) among the groups of hepatocytes (FIG. 5A). However, several monounsaturated and polyunsaturated fatty acids such as the oleic acid (18:1n-9) and mead acid (20:3n-9) were markedly increased in hepatocytes of the N308K group, and to a lesser extent in hepatocytes of the N308K/A313S group when compared to CTL (empty) and SIRT6 WT (FIG. 5A-C).

### Glycerolipids

Regarding glycerolipids, almost not changes were detected in diglyceride or triglyceride levels with the exception of a decrease of several unsaturated species in the WT-transfected hepatocytes when compared to the empty vector group, especially in species with longer acyl chains. This can be easily visualized in the carbon plots displayed in FIGS. 11A and 11B, which represent the influence of the number of carbons and double bond content in the increment or decrement of diglyceride and triglycerides in the WT group compared to the empty vector control group. Some of these triglycerides with longer acyl chains were also reduced in the N308K/A313S group when compared to control hepatocytes (FIG. 1C).

A reduction in the levels of most glycerophospholipids was found in the cells transfected with the WT SIRT6 sequence when compared to the empty vector (FIG. 7), although almost no changes were detected in the culture media (FIG. 8). However, an increment in lysophosphatidylethanolamines species (LPE) was detected for the comparisons between the mutant groups and the WT-transfected hepatocytes, although a higher number of species were altered in the group N308K than in the N308K/A313S (FIG. 5D-I).

### Sphingolipids

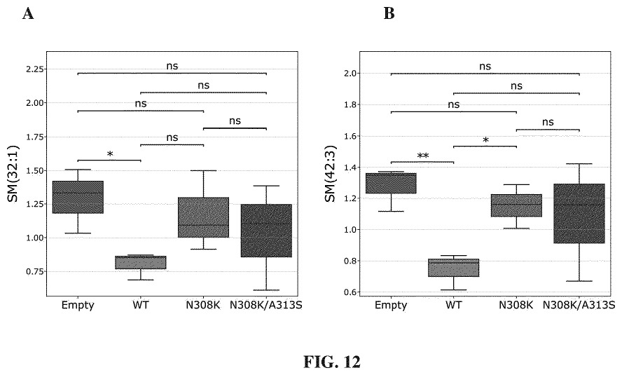

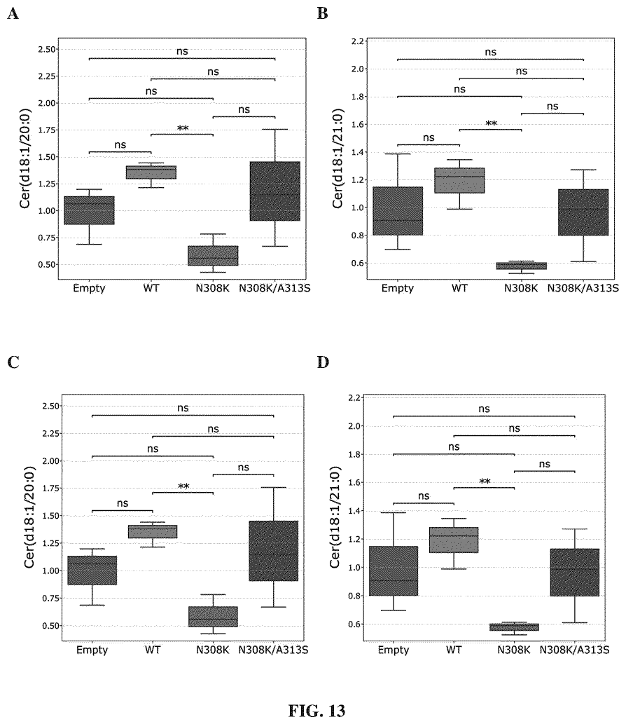

Regarding sphingolipids (ceramides and sphingomyelins), several sphingomyelins were reduced in WT-transfected hepatocytes when compared to the empty-vector group, but there were not differences in their levels when mutants and empty groups were compared (FIG. 12). Ceramides tended to be increased in WT-transfected hepatocytes when compared to the empty-vector group, but only the Cer(d18:1/22:0) reached a p-value <0.05 (FIG. 13). In addition, a reduction in ceramides was only detected in N308K group when compared to the WT group, but not for N308K/A313S group (FIG. 13).

### Conclusion

Altogether our data reveal profound metabolomics changes in IHH upon overexpression of SIRT6 WT and centenarian-associated mutants (N308K and N308K/A313S), compared to control cells. To summarize: (1) almost the complete profile of amino acids was increased in the mutant groups when compared to the hepatocytes transfected with the WT sequence. It was noteworthy the increment of citrulline levels in IHH from the N308K/A313S group; (2) an increment in several unsaturated fatty acid and glycerophospholipids was also detected for N308K and N308K/A313S, although a higher number of species were altered in the former; (3) Ceramides tend to be increased in WT transfected hepatocytes when compared to the control empty vector group. As well, a reduction in ceramides was detected in N308K group when compared to the WT hepatocytes, but not for N308K/A313S group; (4) almost no changes were found in the levels of diglyceride or triglyceride levels.

### Example 3: Overexpression of Centenarian Variant (N308K/A313S) of SIRT6 Inhibits Collagen Deposition and Fibrotic Gene Expression in 3D Spheroids Formed by the Co-Culture of IHH and Human Hepatic Stellatecells

### Material and Methods

### Cell Culture

LX2 cell line was obtained from CLS-GmbH (Eppelheim, Germany). The cell line was cultured in High Glucose DMEM (1×) supplemented with 10% fetal bovine serum (FBS) and 1% penicillin/streptomycin at 37° C. and 5% CO2. The cell culture medium was changed every 2 days and the cells were subcultured using TrypLE Express when reaching 90% confluence.

### 3D Spheroids

For the generation of the cell spheroids, cells were seeded into 96-well round bottom ultra-low attachment plates (BIOFLOAT, faCellilate) at 10000 viable cells per well. Each IHH LV transfect cell line (EMPTY, WT, N308K and N308K/A313S) and normal IHH (control CTL) was co-cultured with LX2 cells with 20:1 ratio, to reproduce the physiological proportion in the liver parenchyma, where hepatocytes are major cell type with only ~5% hepatic stellate cells. The spheroids were grown in DMEM media supplemented as described above. The plates were incubated for five days at 37° C. in a humidified atmosphere of 5% CO2.

### Microscopy and Fluorescence Imaging

The spheroids were fixed with 4% PFA for 10 minutes directly on the cultivation plates and then transferred in mini-tubes. After wash in PBS the spheroids were kept in sucrose 15% for 1 hour, embedded in tissue freezing media (OCT) and then cut to 7 m at −20° C. with a cryotome (Leica Microsystems) and stored at −80° C. for further use. To evaluate the effect of SIRT6 variants and their overexpression on liver tissue fibrosis, spheroid histological sections were immunolabeled to detect Collagen 1A. Slides were washed once in 1×PBS to dissolve the OCT and blocked in 1×PBS supplemented with 0.2% Tween-20 and 5% BSA.

Primary antibody rabbit anti-Collagen I (1:500, ab34710, Abcam) was diluted in DAKO antibody diluent (S202230-2, Agilent technologies) and incubated O.N. in humid chamber at room temperature. After three washes with 1×PBS, mix of secondary antibody (1:500) donkey anti-rabbit IgG coupled with Alexa Fluor™ 647 was applied and incubated for at least 1 h. After three washes in 1×PBS, slides were counterstained with DAPI (1 μg/ml) solution for 15 minutes and mounted in water based hardening media (Mowiol). After hardening (overnight at 4° C.), images were captured using an Axioscan Z.1 (ZEISS) equipped with a Hamamatsu ORCA-Flash 4.0 camera and ImageJ software (NIH, USA) analysis program was used to evaluate all immunofluorescence images. Fibrosis, determined as collagen 1A abundance in spheroid samples, was evaluated as the % of the total spheroid area delineated by DAPI fluorescence at 100× magnification, when at least 5 spheroids per each condition/cell line were used in three consecutive and independent experiments.

### Soluble Collagen Measurement

The spheroids conditioned media (CM) was collected and centrifuged at 1000× g. The cell solution was homogenized on ice using a pre-chilled Dounce homogenizer. Following overnight incubation, the acidic solution was centrifuged at 10,000×g for 15 min at 4° C. to pellet any debris and the clarified supernatant was transferred to a new microfuge tube. Collagen concentration was measured using the Soluble Collagen Assay Kit® (ab241015, Abcam, Cambridge, UK) according to manufacturer's instruction. The fluorescence was measured at an excitation wavelength of 360 nm and an emission wavelength of 460 nm using an Agilent BioTek FLx800 microplate reader.

### Quantitative Real Time PCR

Briefly, column separation technique was used for mRNA isolation with a RNeasy mini-Kit (74106, Qiagen, Germany), according to manufacturer's instructions. At least 4 biological replicates were prepared for each treatment group. Total RNA was quantified on NanoDrop 1000 spectrophotometer (ThermoFisher Scientific) and 1 g of total isolated RNA was used to prepare cDNA using a High-Capacity cDNA Reverse Transcription Kit (4368814, ThermoFisher Scientific). Real Time-PCR was performed with at least two technical replicates using a StepOnePlus™ Real-Time PCR System (Applied Biosystems) and SYBR™ Select Master Mix (4472908, ThermoFisher Scientific). The PCR reaction was held in 10 ul volume and 250 ng of cDNA was added to each well. The primer sequences used in this study are listed in the Table 1.

TABLE 1
Primer sequences used in the study

| | | SEQ |
|---|---|---|
| | | ID |
| Gene | Sequence (5′-3′) | NO: |
| αSMA | F: AAAAGACAGCTACGTGGGTGA | 9 |
| | R: GCCATGTTCTATCGGGTACTTC | 10 |
| COL1A1 | F: GTGCGATGACGTGATCTGTGA | 11 |
| | R: CGGTGGTTTCTTGGTCGGT | 12 |
| TIMP1 | F: ACCACCTTATACCAGCGTTATGA | 13 |
| | R: GGTGTAGACGAACCGGATGTC | 14 |
| VIMENTIN | F: AGTCCACTGAGTACCGGAGAC | 15 |
| | R: CATTTCACGCAATCTGGCGTTC | 16 |
| MMP2 | F: TACAGGATCATTGGCTACACACC | 17 |
| | R: GGTCACATCGCTCCAGACT | 18 |
| GADPH | F: GGTGCGTGCCCAGTTGA | 19 |
| | R: TACTTTCTCCCCGCTTTTT | 20 |

### Results

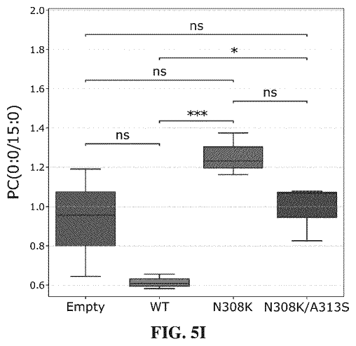

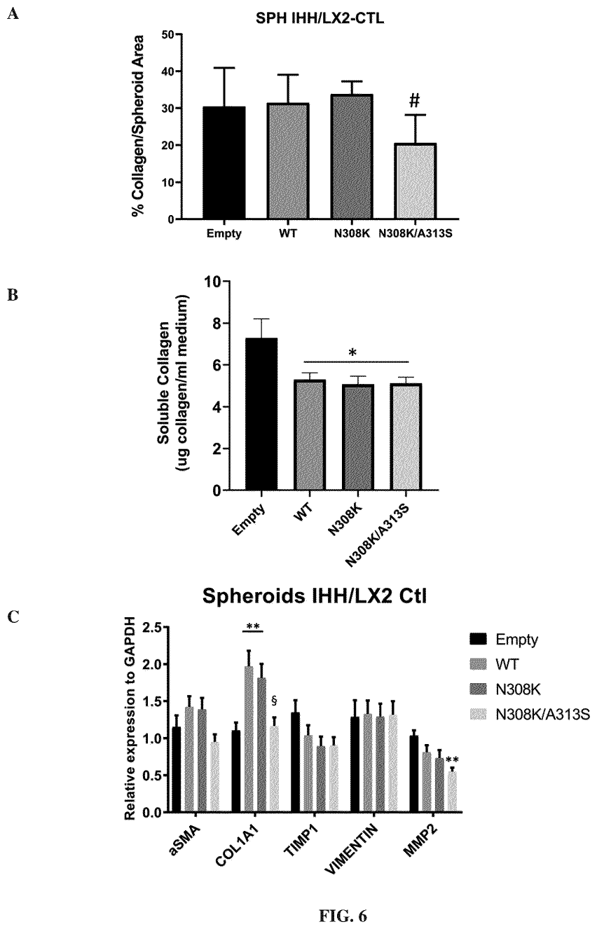

The liver parenchyma is composed of various cell types: while hepatocytes make about 80% of total liver mass, the second most abundant hepatic cell type is represented by hepatic stellate cells (HSC), which account for 5-8%. The crosstalk between these two major hepatic cell types, and HSC-mediated collagen deposition largely controls the progression of fibrosis and inflammation in NAFLD/NASH. Therefore, our metabolomics data led us to investigate the in vitro interaction of the two major hepatic cell types, adopting a 3D spheroid culture model of IHH overexpressing or not SIRT6 and its longevity-associated variants together with hepatic stellate cells (LX2). The spheroid culture allows intercellular connections and communications, reproducing an environment closer to in vivo condition compared to monolayer cell culture IHH and LX2 were co-cultured for 5 days in ultra-low attachment 96 plates and then harvest and processed for the analyses. FIG. 6A shows quantification analysis of the spheroids sections with DAPI (nuclei) and COL1A1 and uncovered a significant decrease in collagen content in the spheroids with IHH overexpressing the N308K/A313S version of SIRT6 compared to all other groups (FIG. 6A). The collagen released in the conditioned media by the spheroids was then measured. Results shows that in all groups overexpressing any of the SIRT6 variants the collagen levels were ~30% lower compared to the vector empty group (FIG. 6B). The mRNA expression of key fibrosis gene markers in the spheroids were also analyzed. The COL1A1 levels were significantly higher in the WT and N308K groups compared to either empty or N308K/A313S groups (FIG. 6C). Moreover, another important marker of fibrosis, MMP2, showed a decreasing trend in all SIRT6 overexpressing groups, with significant lower levels in N308K/A313S group, compared to empty group. Therefore, centenarian-associated SIRT6 variants confer basal antifibrotic effects in in vitro multilineage 3D hepatic spheroids.

### Example 4: In Vitro Assay—Fibrosis

### MATERIAL AND METHODS

### Cell Lines

LX2 human hepatic stellate cell line was obtained from CLS-GmbH (Eppelheim, Germany) and were cultured in high glucose (4.5 g/l) DMEM (1×) supplemented with 10% fetal bovine serum (FBS), 15 mM Hepes buffer (Biowest, France), glutamine, 1% penicillin/streptomycin solution, and 100 μg/ml Normocin at 37° C. and 5% CO₂.

Immortalized human hepatocytes (IHH) Human hepatocyte cell line (IHH), isolated and immortalized by lentiviral transduction with the SV40T antigen and hTERT as previously described [De Gottardi A, Vinciguerra M et al., Lab Invest 2007], were maintained in phenol red-free Dulbecco's modified Eagle's medium (DMEM/F-12) containing 1×10-6 M dexamethasone, 1×10-12 M human insulin (Humalog, Lilly) 10% FBS, and 1% penicillin/streptomycin.

Human primary hepatic stellate cells from a healthy donor (n=1) and from donors with NASH (n=2) were obtained from Lonza were cultured in Human Stellate Cell Growth Media (Catalog #: MCST250) supplemented with 10% fetal bovine serum (FBS), 1% penicillin/streptomycin solution, at 37° C. and 5% CO₂.

AAV2/5 Transduction

The cells, after reaching confluence, were split and seeded into 24-well plate with growth surface area of 2 cm2, where 50×10*4 cells per well were seeded. Immediately after seeding, cells were transduction with AAV2/5 containing SIRT6 constructs: AAV-LUC (luciferase), AAV-SIRT6(WT) and SIRT6(N308K/A313S) (centenarian) at 10*6 vg, in basal DMEM media for the period of 24 h, then the fresh medium was added and cells were cultivated for another 96 h.

qPCR

Column separation technique was used for mRNA isolation with a RNeasy mini-Kit (Qiagen, Germany). At least 4 biological replicates were prepared for each treatment group. Total RNA was quantified on NanoDrop 1000 spectrophotometer, and 1 g of total isolated RNA was used to prepare cDNA using a high-capacity cDNA Reverse Transcription Kit (ThermoFisher Scientific). Real-time PCR was performed with at least two technical replicates using a StepOnePlus™ Real-Time PCR System and SYBR™ Select Master Mix. The PCR reaction was held in 10 μl volume and 250 ng of cDNA was added to each well. GeNorm was used for accurate normalization of qPCR data by geometric averaging of 2 internal control genes (actin, GAPDH).

### Immunoblotting

Cells were harvested from using TrypLE Express and washed with 1×PBS and centrifuged at 300 g. Supernatant was discarded, and the obtained pellet was resuspended in 1×RIPA lysis buffer supplemented with Halt™ Protease and Phosphatase Inhibitor Cocktail (100×, ThermoFisher) and lysed on ice (4° C.) for 30 min with vigorous vortexing every 10 min. The samples were then centrifuged at 10000 g for 10 min at 4 25° C., supernatant was transferred to new microfuge tube, and concentration of protein was measured by Pierce™ BCA Protein Assay Kit (23,225, ThermoFisher) according to manufacturer's instruction. Equal amount of protein samples (at least 20 μg) was mixed with 1× Laemmli Sample buffer (1,610,747, 4×, Bio-Rad), and after heating at 95° C. for 5 min and cooling on ice, equal volumes of proteins (40 μl) were loaded on 10% Mini-PROTEAN® TGX Stain-Free™ Protein Gels (4,568,034, Bio-Rad) and separated by electrophoresis running at 120 V for 45 min. Protein transfer was performed on PVDF membranes using Trans-Blot Turbo RTA Mini 0.45 μm LF PVDF Transfer Kit (1,704,274, Bio-Rad) and Bio-Rad Trans-Blot Turbo Transfer System at 1.3 A and 25 V for 10 min. Membranes were then blocked with 5% bovine serum albumin (BSA, P6154, BioWest) dissolved in TBST buffer (20 mM Tris-HCl, pH 7.6, 140 mM NaCl, 0.1% Tween 20) for at least 30 min and incubated with the specific primary antibodies (see below) diluted in TBST blocking solution, at appropriate dilutions. Following three washes in TBST buffer, membranes were incubated with secondary antibodies conjugated with horseradish peroxidase diluted in TBST blocking buffer. After three further washes with TBST, protein levels were detected by Clarity Western ECL Substrate (1,705,061, Bio-Rad), and the signal detected on Bio-Rad ChemiDoc XRS+imaging systems. For quantitative measurement, the scanned membranes were analyzed using the Image Lab™ Software (Bio-Rad).

### Spheroids

For the generation of the cell spheroids, cells were seeded into 96-well round-bottom ultra-low attachment plates (BIOFLOAT, faCellilate) at 10,000 viable cells per well. Each IHH AAV transfect cell line (LUC, SIRT6wt, SIRT6cent) was co-cultured with LX2 cells with 20:1 ratio, to reproduce the physiological proportion in the liver parenchyma, where hepatocytes are major cell type with only ~5% hepatic stellate cells. The spheroids were grown in DMEM media supplemented as described above. The plates were incubated for 5 days at 37° C. in a humidified atmosphere of 5% CO2. In a subset of spheroids, TGFbeta (10 ng/ml) was added for the last 48 hours of incubation.

### Spheroids Immunofluorescence Analysis

The spheroids were fixed with 4% PFA for 10 min directly on the cultivation plates and then transferred in mini-tubes. After being washed in PBS, the spheroids were kept in sucrose 15% for 1 h, embedded in tissue freezing media (OCT), and then cut to 7 μm at −20° C. with a cryotome (Leica Microsystems) and stored at −80° C. for further use. To evaluate the effect of SIRT6 variants and their overexpression on liver tissue fibrosis, spheroid histological sections were immunolabeled to detect collagen 1A. Slides were washed once in 1×PBS to dissolve the OCT and blocked in 1×PBS supplemented with 0.2% Tween-20 and 5% BSA. Primary antibody rabbit anti-collagen I (1:500, ab34710, Abcam) was diluted in DAKO antibody diluent (S202230-2, Agilent technologies) and incubated O.N. in humid chamber at room temperature. After three washes with 1×PBS, mix of secondary antibody (1:500) donkey anti-rabbit IgG coupled with Alexa Fluor™ 647 was applied and incubated for at least 1 h. After three washes in 1×PBS, slides were counterstained with DAPI (1 μg/ml) solution for 15 min and mounted in water-based hardening media (Mowiol). After hardening (overnight at 4° C.), images were captured using an Axioscan Z.1 (ZEISS) equipped with a Hamamatsu ORCA-Flash 4.0 camera, and ImageJ software (NIH, USA) analysis program was used to evaluate all immunofluorescence images. Fibrosis, determined as collagen 1A abundance in spheroid samples, was evaluated as the % of the total spheroid area delineated by DAPI fluorescence at 100× magnification, when at least 5 spheroids per each condition/cell line were used in three consecutive and independent experiments.

### Results

TABLE 2
Genes/Proteins involved in fibrosis

| Gene | Full name | Role |
|---|---|---|
| αSMA/ | alpha-Smooth muscle | Associated with TGF-β pathway, enhances contractile properties of hepatic |
| Acta2 | actin / Actin alpha 2 | stellate cells leading to liver fibrosis and cirrhosis |
| TIMP1 | Tissue Inhibitor of | TIMP1 is an inhibitory molecule that regulates matrix metalloproteinases |
| | metalloproteinase 1 | (MMPs). In regulating MMPs, TIMP1 plays a crucial role in extracellular |
| | | matrix (ECM) composition |
| VIM | Vimentin | Key factor involved in the progression of liver fibrosis |
| MMP2 | Matrix | Peptidases involved in degradation of the extracellular matrix. Involved |
| | Metalloproteinase 2 | into the regulation of liver fibrogenesis. |
| FN1 | Fibronectin 1 | Protects for liver fibrosis by controlling the availability of active TGF-β |
| | | in the injured liver, which impacts the severity of the resulting fibrosis |
| LOXL2 | Lysyl oxidase-like 2 | Enzyme that promotes the network of collagen fibers of the extracellular |
| | | matrix |
| PDGFRb | platelet-derived | Involved in ECM production and hepatic fibrosis |
| | growth factor | |
| | receptor b | |
| TP63 | Tumor Protein 63 | p63 plays a crucial role in the development and maintenance of epithelial |
| | | tissues and is involved in the regulation of cell cycle arrest and apoptosis. |
| | | TP63 was identified as potential player by RNAseq. |
| COL1A1 | Collagen type 1 | Component of the extracellular matrix, upregulated in liver fibrosis |
| | alpha 1 | |
| COL3A1 | Collagen type 3 | Component of the extracellular matrix, upregulated in liver fibrosis |
| | alpha 1 | |
| COL4A1 | Collagen type 4 | Component of the extracellular matrix, collagen sub-type the most |
| | alpha 1 | expressed in hepatocellular carcinoma, final stage of NASH |
| COL5A1 | Collagen type 5 | Component of the extracellular matrix, upregulated in liver fibrosis |
| | alpha 1 | |
| COL6A1 | Collagen type 6 | Component of the extracellular matrix, upregulated in liver fibrosis and |
| | alpha 1 | indicator of early architectural remodeling in liver fibrosis |

### LX-2 Cells

The liver parenchyma is composed of various cell types: while hepatocytes make about 80% of total liver mass, the second most abundant hepatic cell type is represented by hepatic stellate cells (HSC), which account for 5-8%. The crosstalk between these two major hepatic cell types and HSC-mediated collagen deposition largely controls the progression of fibrosis and inflammation in NAFLD/NASH.

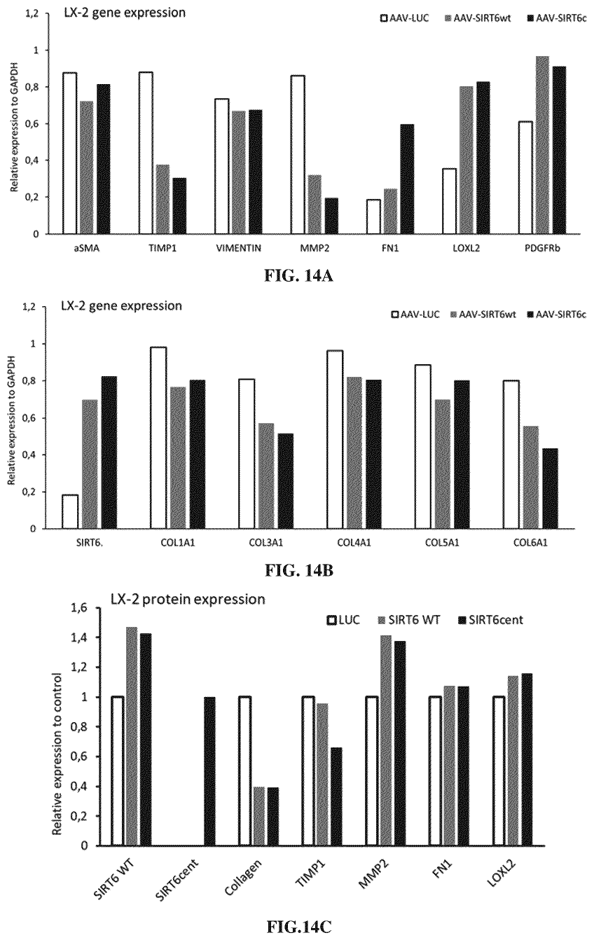

Expression of several genes implicated in liver fibrosis (see Table 2) were measured by qPCR (vimentin, aSMA, TIMP1, MMP2, FN1, LOXL2, PDGFRb, Collagens (COL1A1, COL3A1, COL4A1, COL5A1, COL6A1) or by immunoblotting (Collagen, TIMP1, MMP2, FN1 and LOXL2) in HSC LX2 cell line. Results are shown in FIG. 14A-14C.

Although several changes in gene expression levels were observed at the mRNA level, the most important results at the protein level analyses show an inhibition of collagen production and an increase in MMP2 (matrix metalloproteinase) in HSC overexpressing STRT6-WT or SIRT6cent, together with a SIRT6cent-dependent specific inhibition of TIMP1. Of note, SIRT6 and SIRT6cent overexpression by AAV was confirmed both at the mRNA and at the protein level in LX2.

The MMP2//TIMP1 imbalance, as well as the modulation of FN1, LOXL2 and Collagen expression, suggests SIRT6-dependent liver antifibrotic effects in vitro, with more marked effects dependent on SIRT6cent.

### Spheroids

The liver parenchyma is composed of various cell types: while hepatocytes make about 80% of total liver mass, the second most abundant hepatic cell type is represented by hepatic stellate cells (HSC), which account for 5-8%. The crosstalk between these two major hepatic cell types and HSC-mediated collagen deposition largely controls the progression of fibrosis and inflammation in NAFLD/NASH.

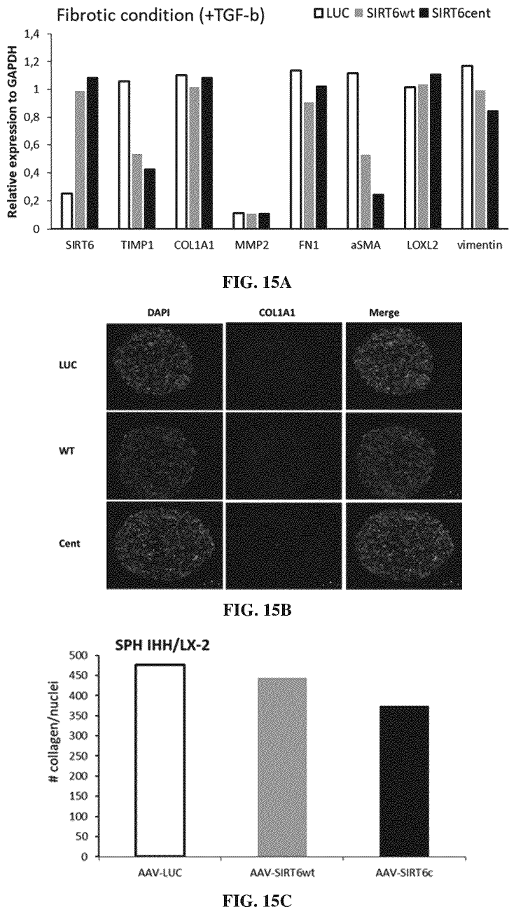

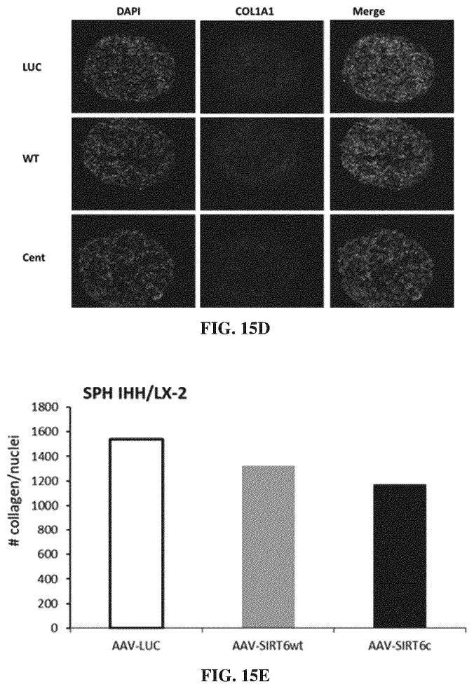

Expression of several genes implicated in liver fibrosis were measured by qPCR (vimentin, αSMA, TIMP1, MMP2, FN1, LOXL2, Collagen (COL1A1) in HSC LX2 cell line. SIRT6 variants overexpression by AAV was confirmed at the mRNA and protein level in LX2 and in spheroids. Results are shown in FIG. 15A-15E.

Although several changes in gene expression levels were observed at the mRNA level, the most important results at the protein level analyses in the spheroids experiments show an inhibition of collagen production in spheroids where IHH were overexpressing SIRT6-WT or SIRT6cent, suggesting anti-fibrotic effects which were more marked upon SIRT6cent overexpression. The antifibrotic effects were observed both in absence and in presence of TGFbeta, a major pro-fibrotic agent.

### Primary Hepatic Stellate Cells

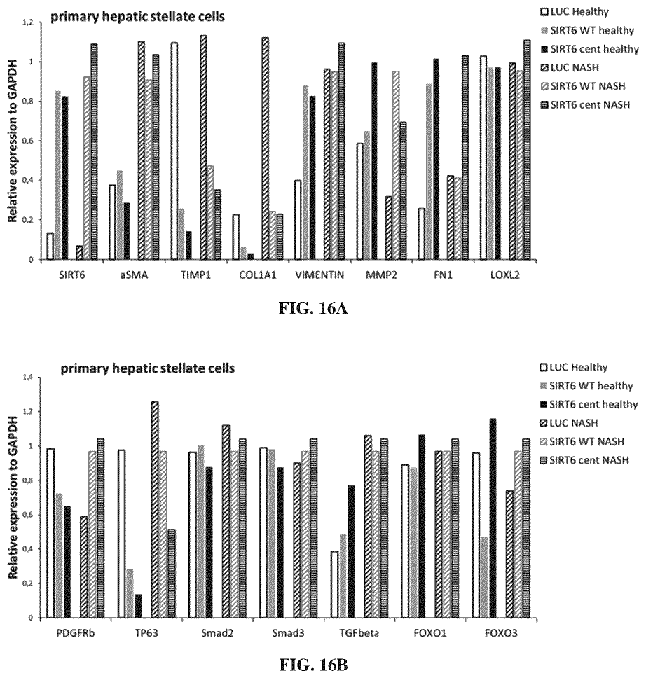

Results are shown in FIG. 16A-16B. Several changes in gene expression levels were observed at the mRNA level. In HSC from NASH patients there was a general activation of profibrotic markers (αSMA, COL1A1, vimentin, TP63, TGFbeta), as expected. As it was observed for the LX2 cells at the protein level, the decrease in collagen and the MMP2//TIMP1 imbalance suggest SIRT6-dependent antifibrotic effects in both healthy and NASH HSC, with more marked effects dependent on SIRT6cent. Interestingly, overexpression of SIRT6wt and, and more markedly regarding SIRT6cent, significantly inhibited the mRNA levels of p63, a newly appreciated pro-fibrotic factor.

Data summarizing fibrosis levels are summarized in Table 3 below.

TABLE 3
Fibrosis level data summary

| | | | | Spheroids | | | | |
|---|---|---|---|---|---|---|---|---|
| Hepatic stellate cells (LX-2) | | | | Transient | | | Primary hepatic | |
| | Transient | | Permanent | expression | | Permanent | stellate cells | |
| | expression | | expression | RNA | RNA | expression | Transient | |
| Gene | RNA | protein | RNA | Basal | fibrotic | RNA | expression | Conclusion |
| aSMA | Minor | NA | ↓ (cent) | Minor | ↓ (cent) | ↓ | NA | ↓ (cent) |
| | ↓ (wt | | | ↑ (wt | | | | |
| | and | | | and | | | | |
| | cent) | | | cent) | | | | |
| TIMP1 | ↓ (wt | ↓ (cent) | / | ↓ (wt | ↓ (wt | ↓ (wt | ↓ (wt | ↓ (wt |
| | and | | | and | and | and | and | and |
| | cent) | | | cent) | cent) | cent) | cent) | cent) |
| Vimentin | Minor | NA | / | / | ↓ (cent) | / | NA | / |
| | ↓ (wt | | | | | | | |
| | and | | | | | | | |
| | cent) | | | | | | | |
| MMP2 | ↓ (wt | ↑ (wt | NA | ↑ (cent) | / | ↓ (SIRT6c) | ↑ | ↑ (wt and |
| | and | and | | | | | | cent) in |
| | cent) | cent) | | | | | | primary |
| | | | | | | | | cells; |
| | | | | | | | | ↓ RNA but |
| | | | | | | | | ↑ protein in |
| | | | | | | | | cell lines |
| FN1 | ↑ (cent) | / | NA | ↑ (cent) | minor ↓ | NA | Healthy: | ↑ (cent) - |
| | | | | | | | ↑ (wt and | only RNA |
| | | | | | | | cent); | |
| | | | | | | | NASH: | |
| | | | | | | | ↑ (cent) | |
| LOXL2 | ↑ (wt | / | NA | ↑ Minor | / | NA | NASH: | ↑ (cent) |
| | and | | | (cent) | | | ↑ Minor | |
| | cent) | | | | | | (cent) | |
| PDGFRb | ↑ (wt | NA | NA | NA | NA | NA | Healthy: | ↑ (wt and |
| | and | | | | | | ↓ (wt and | cent) in |
| | cent) | | | | | | cent); | LX2 and |
| | | | | | | | NASH: | NASH |
| | | | | | | | ↑ (wt and | primary |
| | | | | | | | cent) | cells; |
| | | | | | | | | ↓ (wt and |
| | | | | | | | | cent) |
| | | | | | | | | in healthy |
| | | | | | | | | primary |
| | | | | | | | | cells |
| TP63 | NA | NA | NA | NA | NA | NA | ↓ | ↓ |
| COL1A1 | ↓ (wt | ↓ (wt | ↓ (cent) | minor | / | ↑ (wt) | ↓ | ↓ |
| | and | and | | ↓ (wt | | and/ | | |
| | cent) | cent) | | and | | (cent) | | |
| | | | | cent) | | | | |
| COL3A1 | ↓ (wt | | NA | NA | | | / | / |
| | and | | | | | | | |
| | cent) | | | | | | | |
| COL4A1 | minor ↓ | | NA | NA | | | / | / |
| COL5A1 | minor ↓ | | NA | NA | | | / | / |
| COL6A1 | ↓ (wt | | NA | NA | | | / | / |
| | and | | | | | | | |
| | cent) | | | | | | | |

### Example 5: In Vitro Assay—Lipid Metabolism

### Material and Methods

### Cell Line

Immortalized human hepatocytes (IHH) Human hepatocyte cell line (IHH), isolated and immortalized by lentiviral transduction with the SV40T antigen and hTERT as previously described [De Gottardi A. Vinciguerra M et al., Lab Invest 2007], were maintained in phenol red-free Dulbecco's modified Eagle's medium (DMEM/F-12) containing 1×10-6 M dexamethasone, 1×10-12 M human insulin (Humalog, Lilly) 10% FBS, and 1% penicillin/streptomycin.

### AAV2/5 Transduction

The cells, after reaching confluence, were split and seeded into 24-well plate with growth surface area of 2 cm2, where 50×10*4 cells per well were seeded. Immediately after seeding, cells were infected with AAV2/5 containing SIRT6 constructs: AAV-LUC (luciferase), AAV-SIRT6(WT) and SIRT6(N308K/A313S) (centenarian) at 10*6 vg, in basal DMEM media for the period of 24 h, then the fresh medium was added and cells were cultivated for another 96 h.

qPCR

Column separation technique was used for mRNA isolation with a RNeasy mini-Kit (Qiagen, Germany). At least 4 biological replicates were prepared for each treatment group. Total RNA was quantified on NanoDrop 1000 spectrophotometer, and 1 g of total isolated RNA was used to prepare cDNA using a high-capacity cDNA Reverse Transcription Kit (ThermoFisher Scientific). Real-time PCR was performed with at least two technical replicates using a StepOnePlus™ Real-Time PCR System and SYBR™ Select Master Mix. The PCR reaction was held in 10 μl volume and 250 ng of cDNA was added to each well. GeNorm was used for accurate normalization of qPCR data by geometric averaging of 2 internal control genes (actin, GAPDH).

### Results

TABLE 4
Genes/Proteins involved in lipids metabolism

| Gene | Full name | Role |
|---|---|---|
| CD36 | Differentiated | Fatty acid translocase, contributes significantly to hepatic steaosis by taking part |
| | cluster 36 | in fatty acid uptake as well as triglyceride storage and secretion. |
| FASN | Fatty acid Synthase | Drives de novo lipogenesis (catalyzes the last step in fatty acid biosynthesis) |
| | | and mediates pro-inflammatory and fibrogenic signaling. |
| FABP5 | Fatty acid | Intracellular chaperone of fatty acid molecules that regulates lipid metabolism |
| | binding protein 5 | and cell growth. Plays also a role in cancer progression. |
| DGAT1 | Diacylglycerol | Key enzyme of Triglycerides synthesis in fatty liver development. |
| | acyltransferase 1 | |
| ACC1 | Acetyl-CoA | Enzyme regulating the de novo lipogenesis flux. Upregulated in the liver of |
| | Carboxylase 1 | patients with nonalcoholic fatty liver disease (NAFLD) |
| SGMS1 | Sphingomyelin | Enzyme involved in the metabolism of Glucosylceramide (GluCer). GluCer |
| | synthase 1 | accelerates liver steatosis, steatohepatitis, and tumorigenesis. Glucosylceramide |
| | | stimulates transforming growth factor beta 1 (TGFβ1) activation, which |
| | | mediates liver fibrosis. Human NASH patients were shown to have higher liver |
| | | GluCer synthase and higher plasma GluCer levels. |

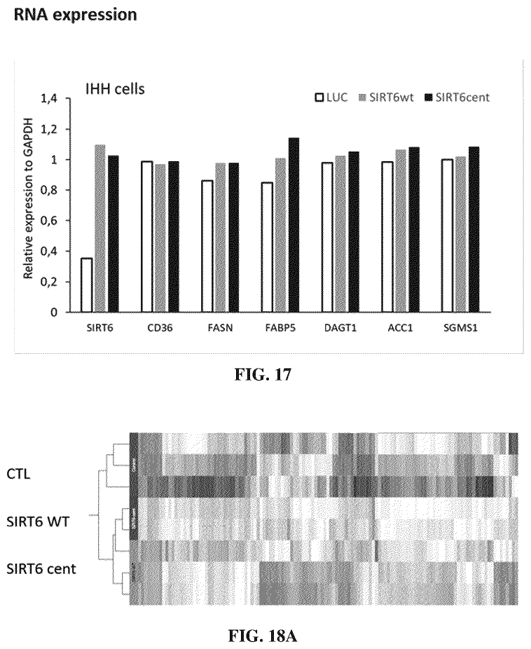

Results are shown in FIG. 17 Expression of several genes implicated in hepatic lipid metabolism (see Table 4) were measured by qPCR (CD36, FASN, FABP5, DAGT1, ACC1, SGMS1) in IHH cell line. SIRT6 variants overexpression by AAV was confirmed at the mRNA level.

qPCR analysis revealed increased levels of genes involved in the regulation of lipids metabolism and NASH progression (FABP5, SGMS1 and ACC), in IHH cells overexpressing SIRT6wt or SIRT6cent when compared to controls.

Data summarizing lipids metabolism are summarized in Table 5 below.

TABLE 5
Lipids metabolim data summary

| IHH | | | Permanent | |
|---|---|---|---|---|
| | Transient expression | | expression | |
| Gene | RNA | protein | RNA | Conclusion |
| CD36 | / | NA | ↓ (wt and cent) | / |
| FASN | ↑ (wt and cent) | NA | ↓ (wt and cent) | / |
| FABP5 | ↑ (cent) | NA | ↑ (wt and cent) | ↑ (cent) |
| DGAT1 | / | NA | ↓ (cent) | / |
| ACC1 | Minor ↑ | NA | / | Minor ↑ |
| | (wt and cent) | | | (wt and cent) |
| SGMS1 | Minor ↑ (cent) | NA | ↑ (cent) | ↑ (cent) |
| CERS1 | NA | NA | Minor ↓ (cent) | / |
| FADS1 | NA | NA | / | / |
| SCD | NA | NA | / | / |

### Example 6: In Vitro Transcriptomics

### Material and Methods

### Cell Line

Immortalized human hepatocytes (IHH) Human hepatocyte cell line (IHH), isolated and immortalized by lentiviral transduction with the SV40T antigen and hTERT as previously described [De Gottardi A, Vinciguerra M et al., Lab Invest 2007], were maintained in phenol red-free Dulbecco's modified Eagle's medium (DMEM/F-12) containing 1×10-6 M dexamethasone, 1×10-12 M human insulin (Humalog, Lilly) 10% FBS, and 1% penicillin/streptomycin.

### AAV2/5 Transduction

The cells, after reaching confluence, were split and seeded into 24-well plate with growth surface area of 2 cm2, where 50×10*4 cells per well were seeded. Immediately after seeding, cells were infected with AAV2/5 containing SIRT6 constructs: AAV-LUC (luciferase), AAV-SIRT6(WT) and SIRT6(N308K/A313S) (centenarian) at 10*6 vg, in basal DMEM media for the period of 24 h, then the fresh medium was added and cells were cultivated for another 96 h.

### RNA-Seq

Indexed libraries were prepared from 2 mg/ea purified RNA from IHH-LUC, IHH-SIRT6wt and IHH-SIRT6cent cells (n=4 for each condition) with the TruSeq Total Stranded RNA Sample Prep Kit (Illumina, Cambridge, UK) according to the manufacturer's instructions. Libraries were quantified using the Agilent 2100 Bioanalyzer (Agilent Technologies, Santa Clara, USA) and pooled so that each index-tagged sample was present in equimolar amounts; the final concentration of the pooled samples was 2 nmol/L. Pooled samples were then subjected to cluster generation and sequencing using an Illumina HiSeq 2500 System (Illumina, Cambridge, UK) in a 2×100 paired-end format at a final concentration of 8 pmol/L. Short reads were aligned against the GRCm38 genome assembly using STAR (ver. 2.5.1a). Piled up reads were counted with htseq-count. Normalization of reads counts and their comparisons were performed using R package. Genes were considered differentially expressed between groups if their expression values differed by more than 2-folds, significantly (q-value ≤0.05). Pathway enrichment analysis was performed by using Ingenuity Pathway Analysis (QIAGEN Inc). All computations were performed with R ver. 3.4.1 (R Core Team 2017).

### Whole Transcriptome Analysis

The whole transcriptome of IHH cells transduced with AAV-SIRT6cent and AAV-SIRT6wt was analyzed using Ingenuity Pathway Analysis (IPA).

### Results

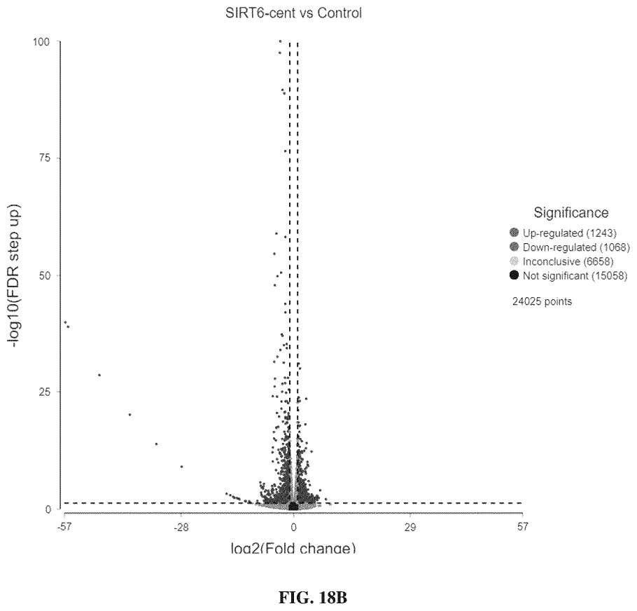

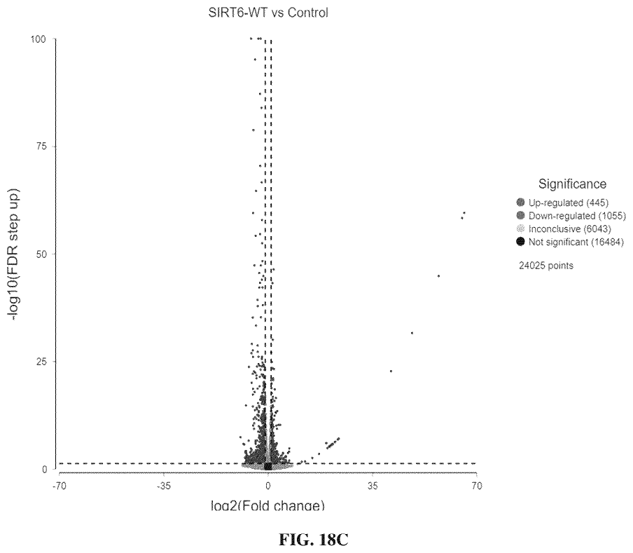

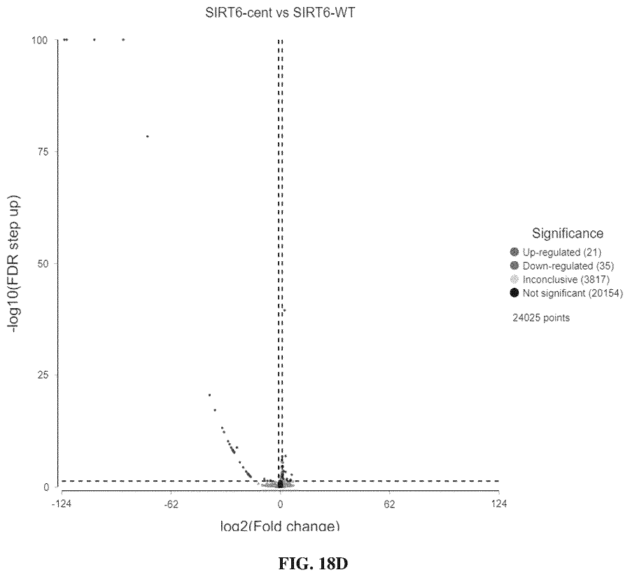

Results are shown in FIG. 18A-18D. Although massive changes in gene expression levels were observed in the comparison between SIRT6wt or SIRT6cent versus the control (LUC) (as evidenced in the heatmap and in the volcano plots), only 56 genes were differentially expressed in the comparison between SIRT6 variant overexpression (SIRT6wt versus SIRT6cent), suggesting a finely-tuned transcriptional control.

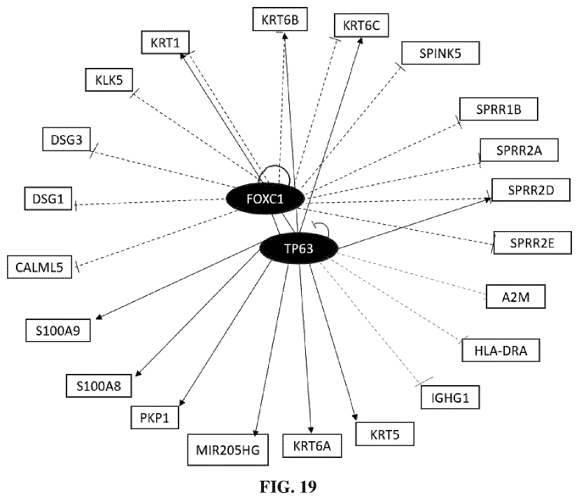

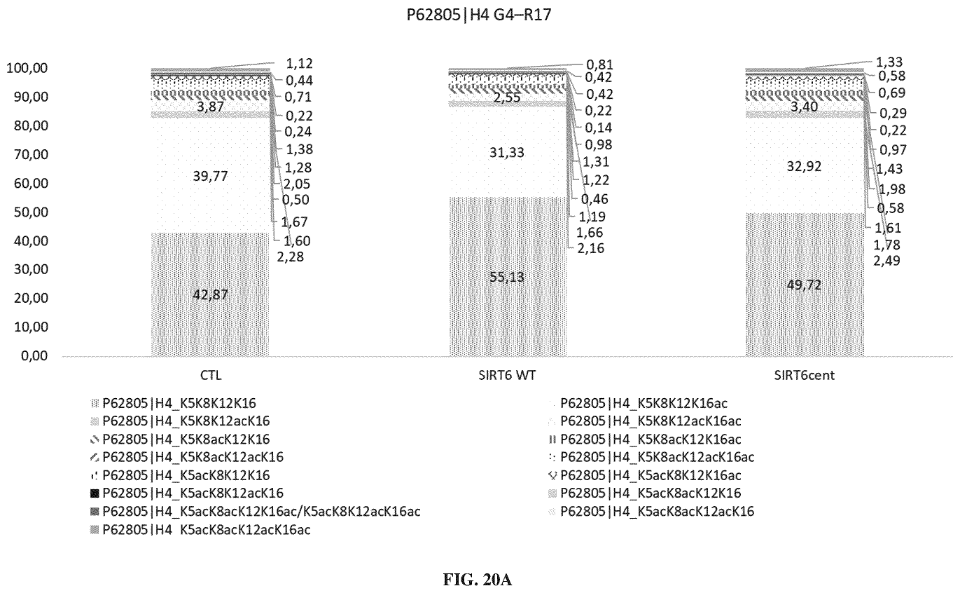

An analysis of pathways differentially expressed between AAV-SIRT6-WT and AAV-SIRT6-Cent treated cells is represented on FIG. 19.

A down-expression of the b-catenin pathway in AAV-SIRT6-Cent vs. AAV-SIRT6-WT treated IHH cells was shown. Of the 21 genes down-regulated in AAV-SIRT6wt vs AAV-SIRT6cent treated cells, 12 genes are involved into the b-catenin pathway (PKP1, S100AB, SPRR2D, KRT1, KRT6C, A2M, KRT6B, KRT5, HLA-DRA, KRT6A, IGHG1, MIR205HG). See Table 6.

TABLE 6
Down-expression of the b-catenin pathway in
AAV-SIRT6-Cent vs. AAV-SIRT6-WT treated cells

| Gene | FC (WT vs Cent) |
|---|---|
| PKP1 | 5.29E+06 |
| S100A8 | 1.05E+07 |
| SPRR2D | 2.39E+01 |
| KRT1 | 2.64E+12 |
| KRT6C | 1.71E+17 |
| A2M | 2.03E+06 |
| KRT6B | 6.52E+19 |
| KRT5 | 4.05E+19 |
| HLA-DRA | 1.55E+01 |
| KRT6A | 5.23E+03 |
| IGHG1 | 3.26E+06 |
| MIR205HG | 7.37E+05 |

TP63 has been identified by the software as a central gene controlling the b-catenin pathway in AAV-treated IHH.

A down-expression of the glucocorticoid pathway in AAV-SIRT6-Cent vs. AAV-SIRT6-WT treated IHH cells was shown. Of the 21 genes down-regulated in AAV-STRT6 wt vs AAV-STRT6cent treated cells, 11 genes are involved into the glucocorticoid pathway (SPINK5, DSG1, SPRR2D, KLK5, KRT1, SPRR1B, KRT6C, CALML5, KRT6B, SPRR2E, SPEE2A). See Table 7.

TABLE 7
Down-expression of the Glucocorticoid pathway in
AAV-SIRT6-Cent vs. AAV-SIRT6-WT treated cells.

| Gene | FC (WT vs Cent) |
|---|---|
| SPINK5 | 1.46E+06 |
| DSG1 | 5.64E+01 |
| SPRR2D | 2.39E+01 |
| KLK5 | 7.28E+05 |
| KRT1 | 2.64E+12 |
| SPRR1B | 1.35E+07 |
| KRT6C | 1.71E+17 |
| CALML5 | 5.96E+06 |
| KRT6B | 6.52E+19 |
| SPRR2E | 1.69E+06 |
| SPRR2A | 6.69E+01 |

FOXC1 has been identified by the software as a central gene controlling the glucocorticoid pathway in AAV-treated IHH.

Of note, for this analysis, the whole transcriptome was used, not only the 56 differentially expressed genes, which however were highlighted in the analysis again as most important genes.

In conclusion, SIRT6cent in involved in the control of b-catenin and glucorticoid pathways in immortalized human hepatocytes.

### Example 7: Posttranslational Modifications (PTM) of Histones by SIRT6 WT and SIRT6cent in 3T3-L1 Adipocytes

The most robust enzymatic activity described for SIRT6 is its function as a histone deacetylase. Strong evidence suggests that SIRT6 is hardwired to target gene promoters and represses gene transcription by removing acetylation on H3K9, H3K18 and H3K56 heterochromatic sites. Of all histone post-translational modification (PTM) types, acetylation and methylation are the two most well-studied types, and they functionally interact to fine tune transcriptional outputs. To gain insight into the SIRT6-dependent epigenomic regulation during adipogenesis, we performed a comprehensive analysis of histone acetylation/methylation PTMs by mass-spectrometry (LC-MS/MS) in 3T3-L1 differentiated adipocytes (AdiE, AdiWT and AdiCent).

### Material and Methods

Histone Extraction from 3T3-L1 Cells Overexpressing LUC (Ctl or AdiE), SIRT6wt (or AdiWT) or SIRT6cent (or AdiCent)

Histone extraction protocol was adapted from previous work [Cincarova, L., et al., A combined approach for the study of histone deacetylase inhibitors. Mol Biosyst, 2012. 8(11): p. 2937-45.]. Six replicates of each sample were carried out. Cells on cultivation plates were slightly washed with cold PBS, collected in lysis buffer (80 mM NaCl, 20 mM EDTA (Bio-Rad, California, USA), 1% Triton X-100 (Carl Roth, Germany), 45 mM sodium butyrate and 0.1 mM PMSF (Thermo Fisher Scientific)), and incubated on ice for 20 min. After centrifugation (20 000 g, 8 min, 4° C.), the upper lipid layer was removed, and PBS was added to the remaining sample containing chromatin.

Additional three washes with PBS were performed (20 000 g, 10 min, 4° C.), and the pellet was resuspended in 250 μL of ice-cold H2SO4 (Penta, Czech Republic) and incubated while shaking at 4° C. for 2 h. Supernatant cleared by centrifugation (20 000 g, 10 min, 4° C.) was diluted with 250 μL of 50% ice-cold trichloroacetic acid and incubated while shaking at 0° C. for 30 min. The resulting precipitate was harvested by centrifugation (20 000 g, 30 min, 4° C.), washed with 50 mM HCl (Penta) in acetone and twice with acetone, and dried at RT. Prepared histone extracts were dissolved in 20 μL of water.

### Chemical Derivatization of Histone Extract

The volume of histone extracts was reduced in vacuum concentrator to 5 μL, 5 μL of acetonitrile (ACN; Honeywell, USA) was added, and the samples were subjected to microwave-assisted histone derivatization using trimethylacetic anhydride (Sequencing grade modified, Promega Corporation, Madison, WI, USA) according to a previously published procedure [48]. The pH was adjusted to 8 with NH4OH and 3 μL of derivatization reagent consisting of trimethylacetic anhydride (Merck Millipore, Burlington, MA, USA), and ACN in a 1:3 (v/v) ratio was added. The samples were incubated for 5 h at RT with shaking, followed by repeated derivatization step including 16 h incubation. Subsequently, samples proceeded two rounds of microwave-assisted histone derivatization, as follows. Samples' volume was reduced to 5 μL in vacuum concentrator, and 50% (v/v) ACN was added to a final volume of 12 μL. Each round included three derivatization sub-cycles consisting of samples' pH adjustment to 8 with NH40H, addition of 3 μL of derivatization reagent, and two 1 min incubation in the microwave oven at 350 W (short spin between incubations). Microtubes with samples were covered with a glass beaker during incubation in the microwave oven. After two complete rounds (6 additions of reagent in total) samples' volume was reduced to 5 μl, and 0.3 μg of SOLu-Trypsin (Merck) in 40 μL of 100 mM ammonium bicarbonate (ABC) was added, and samples were incubated at 37° C. for 4 h followed by another addition of 0.3 μg of SOLu-Trypsin for 12 h incubation. Digested samples underwent two rounds of the above-described microwave-assisted derivatization for labeling newly released peptide N-termini. After the first round, samples were diluted to a final volume of 24 μL and completely dried out after the second round. Derivatized histones were diluted with 0.1% TFA and desalted on Pierce C18 Spin Tips #84850 (Thermo Fisher Scientific). Peptides were eluted sequentially with 0.1% TFA in 50% ACN and 0.1% TFA in 75% ACN. Samples were dried in a vacuum concentrator to remove TFA and reconstituted in 0.1% FA (Honeywell) before LC-MS/MS analysis.

### LC-MS/MS and Database Search of Histone Peptides

Chemically derivatized peptides were measured using LC-MS/MS consisting of an Ultimate 3000 RSLC-nano system coupled to an Orbitrap Lumos Tribrid spectrometer (Thermo Fischer Scientific) equipped with a Digital PicoView 550 ion source (New Objective) and Active Background Ion Reduction Device (ESI Source Solutions). Prior to LC separation, tryptic digests were online concentrated on Precolumn C18 PepMap100 trap column (5 μm particles, 300 μm ID, 5 mm; Waters). Chromatographic separation was performed on Aurora C18 analytical column (1.6 m particles, 75 μm ID, 25 mm; Ion Opticks). The mobile phase consisted of 0.1% v formic acid in water (A) and 0.1% formic acid in 80% ACN (B), with the following proportions of B: 5% to 25% (0-20 min), 25 to 29% (20-30 min), 29 to 32% (30-40 min), 32 to 38% (40-55 min), 38 to 50% (55-75 min), and 50 to 85% (75-85 min), followed by isocratic wash of 85% B (85-95 min). Equilibration with 99:1 (mobile phase A:B; flow rate 500 nl/min) of the trapping column and the column was done prior to sample injection to the sample loop. The analytical column outlet was directly connected to the ion source. MS data were acquired using a data-dependent strategy, selecting up to top 10 precursors based on precursor abundance in a survey scan (m/z 350-2000). The resolution of the survey scan was 60000 with a target value of 4×105, one microscan and maximum injection time of 54 ms. HCD MS/MS spectra were acquired with a target value of 5×104 and resolution of 15000. The maximum injection time for MS/MS was 22 ms. Dynamic exclusion was enabled for 60 s after one MS/MS spectrum acquisition and early expiration was disabled. The isolation window for MS/MS fragmentation was set to 1.6 m/z.

### Evaluation of Mass Spectrometry Data

The raw mass spectrometric data files were analyzed using Proteome Discoverer software (Thermo Fisher Scientific; version 2.2.0.388) with in-house Mascot search engine (Matrix Science, version 2.6.2) to compare acquired spectra with entries in the UniProtKB human database (version 2021_12; 20594 protein sequences), cRAP contaminant database, and in-house histone human database (version 2019_10; 52 protein sequences). Mass tolerances for peptides and MS/MS fragments were 10 ppm and 0.03 Da (0.5 Da for cRAP), respectively. Semi-Arg-C for enzyme specificity allowing up to two missed cleavages was set. For searches against cRAP database, the variable modification settings were oxidation (M), deamidation (N, Q), acetylation (K) and trimethylacetylation (K, N-term, S, T, Y). For searches against UniProtKB human databases, they were trimethylacetylation (K, N-term, S, T, Y). For histone database searches they were acetylation (K), methylation (K, R), dimethylation (K), trimethylation (K), phosphorylation (S, T), and trimethylacetylation (K, N-term, S, T, Y). Selected histone peptide identifications were manually verified and quantified from the peak areas derived from the EICs using Skyline (64-bit, v. 23.1.1.268 software), including identification alignment across the raw files based on retention time and m/z.

The relative abundances of histone peptides were evaluated according to previously published methodology using R script in KNIME Analytics Platform. The relative abundance of a particular modified peptide form was calculated from the ratio of each precursor peak area to the total area of the respective peptide sequence. The peak areas corresponding to post-translationally modified forms of individual histone peptide were treated as compositions and Aitchison's methodology based on log-ratios was applied in the statistical evaluation. First, the missing values were imputed by iterative least trimmed squares regression and areas were transformed to relative abundances (percentages). In order to evaluate global acetylation or methylation, acetylated and non-acetylated forms, and analogically methylated and non-methylated forms of each peptide were amalgamated. These amalgamated abundances were then ilr-transformed and compared by Hotelling's T2 test to globally assess the differences in their distribution. For comparison of all individual peptide forms, for each peptide, the log 2 ratio of relative abundance of one form to the sum of relative abundances of all other forms (alr-transformation of a 2-part composition) was calculated and the t-test was applied to assess the difference in each individual form. Note that in compositional data, the relative abundances of individual parts were not directly comparable due to the constant sum constraint leading to a spurious negative correlation. The data analysis was performed in R version 3.6.3 using the compositions and Hotelling R packages for ilr and alr transformations, and Hotelling T2 test, respectively. Hotelling: Hotelling's T\hat{ }2 Test and Variants. R package version 1.0-5.

### Results

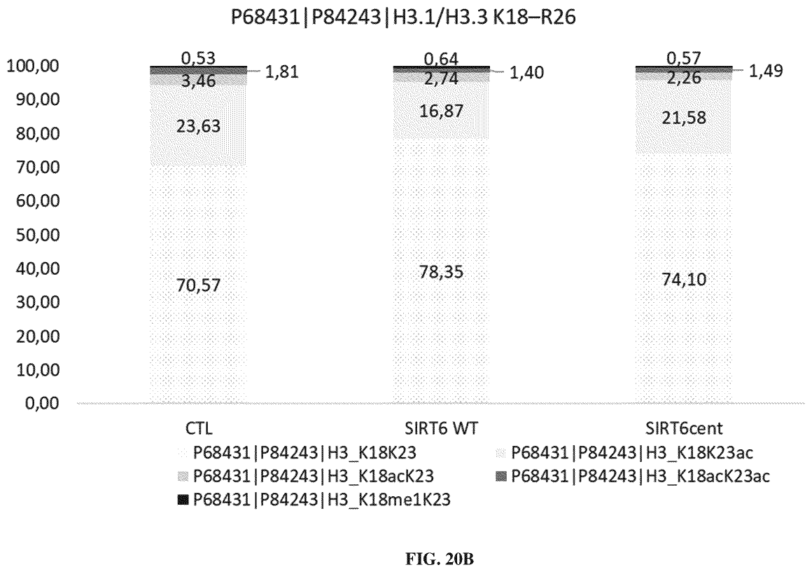

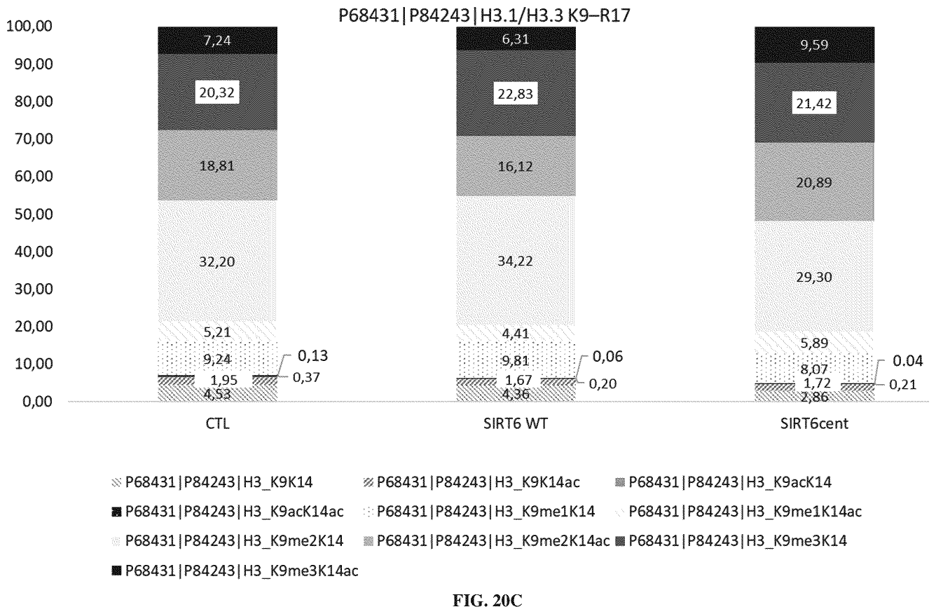

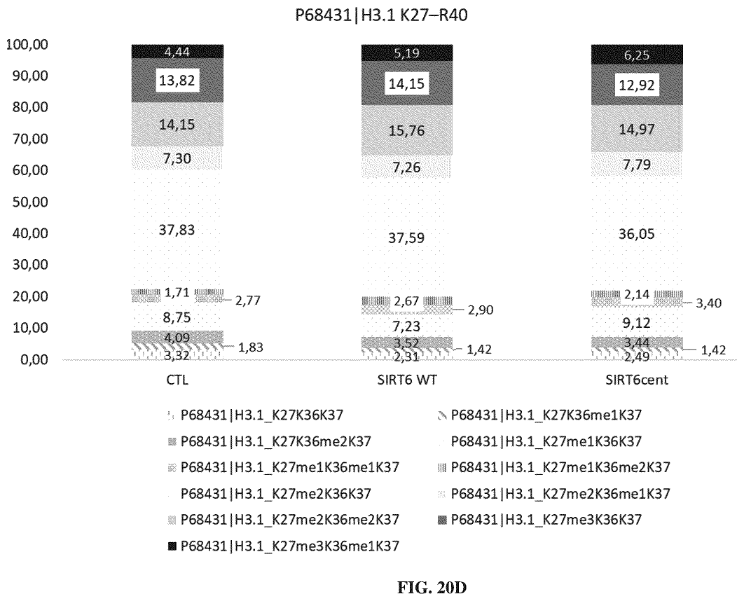

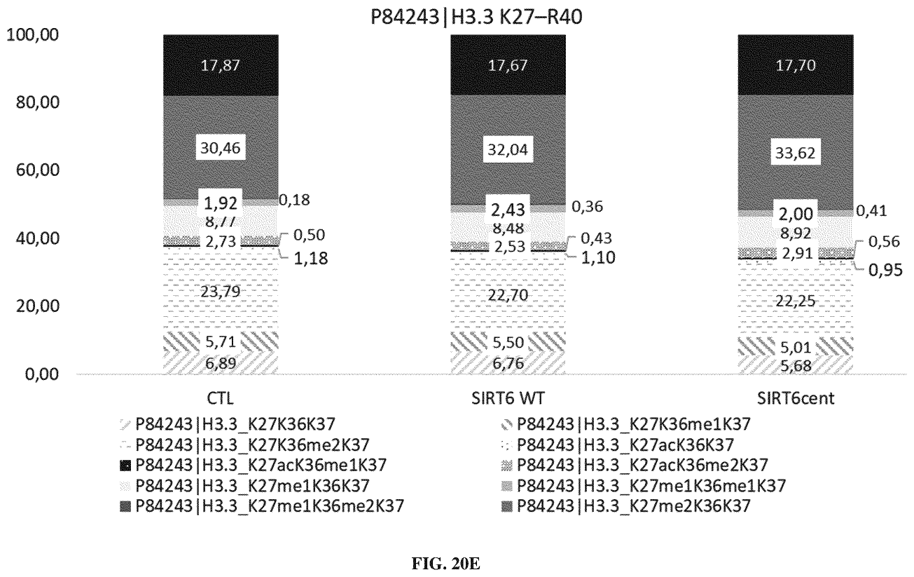

Results are shown on FIG. 20A and Table 8 for HISTONE H4 G4KGGKGLGKGGAKR17, in FIG. 20B and Table 9 for HISTONE H3.1/H3.3 K18QLATKAAR26, in FIG. 20C and Table 10 for HISTONE H3.1/H3.3 K9STGGKAPR17, in FIG. 20D and Table 11 for HISTONE H3.1 K27SAPATGGVKKPHR40, in FIG. 20E and Table 12 for HISTONE H3.3 K27SAPSTGGVKKPHR40.

TABLE 8

| | CLT vs | CTL vs | CTL vs |
|---|---|---|---|
| row ID | SIRT6cent | SIRT6 WT | SIRT6 WT |
| sp\|P62805\|H4_K5K8K12K16 | 0.021 | 0.078 | 0.012 |
| sp\|P62805\|H4_K5K8K12K16ac | 0.001 | 0.291 | 0.004 |
| sp\|P62805\|H4_K5K8K12acK16 | 0.452 | 0.260 | 0.094 |
| sp\|P62805\|H4_K5K8K12acK16ac | 0.167 | 0.052 | 0.023 |
| sp\|P62805\|H4_K5K8acK12K16 | 0.364 | 0.597 | 0.637 |
| sp\|P62805\|H4_K5K8acK12K16ac | 0.414 | 0.008 | 0.013 |
| sp\|P62805\|H4_K5K8acK12acK16 | 0.378 | 0.208 | 0.259 |
| sp\|P62805\|H4_K5K8acK12acK16ac | 0.714 | 0.012 | 0.014 |
| sp\|P62805\|H4_K5acK8K12K16 | 0.082 | 0.192 | 0.683 |
| sp\|P62805\|H4_K5acK8K12K16ac | 0.349 | 0.987 | 0.006 |
| sp\|P62805\|H4_K5acK8K12acK16 | 0.482 | 0.033 | 0.020 |
| sp\|P62805\|H4_K5acK8acK12K16 | 0.057 | 0.048 | 0.799 |
| sp\|P62805\|H4_K5acK8acK12K16ac/ | 0.743 | 0.038 | 0.041 |
| K5acK8K12acK16ac | | | |
| sp\|P62805\|H4_K5acK8acK12acK16 | 0.089 | 0.111 | 0.764 |
| sp\|P62805\|H4_K5acK8acK12acK16ac | 0.180 | 0.035 | 0.100 |

TABLE 9

| | CTL vs | SIRT6 WT vs | CTL vs |
|---|---|---|---|
| row ID | SIRT6cent | SIRT6cent | SIRT |
| sp\|P68431\|H31_K18K23 | 0.012 | 0.049 | 0.013 |
| sp\|P68431\|H31_K18K23ac | 0.027 | 0.027 | 0.010 |
| sp\|P68431\|H31_K18acK23 | 0.003 | 0.038 | 0.014 |
| sp\|P68431\|H31_K18acK23ac | 0.089 | 0.551 | 0.060 |
| sp\|P68431\|H31_K18me1K23 | 0.493 | 0.283 | 0.058 |

TABLE 10

| | CTL vs | SIRT6 WT vs | CTL vs |
|---|---|---|---|
| row ID | SIRT6cent | SIRT6cent | SIRT6 |
| sp\|P68431\|H31_K9K14 | 0.007 | 0.111 | 0.841 |
| sp\|P68431\|H31_K9K14ac | 0.184 | 0.830 | 0.342 |
| sp\|P68431\|H31_K9acK14 | 0.000 | 0.012 | 0.001 |
| sp\|P68431\|H31_K9acK14ac | 0.001 | 0.379 | 0.000 |
| sp\|P68431\|H31_K9me1K14 | 0.025 | 0.021 | 0.318 |
| sp\|P68431\|H31_K9me1K14ac | 0.006 | 0.004 | 0.011 |
| sp\|P68431\|H31_K9me2K14 | 0.002 | 0.002 | 0.038 |
| sp\|P68431\|H31_K9me2K14ac | 0.014 | 0.007 | 0.018 |
| sp\|P68431\|H31_K9me3K14 | 0.045 | 0.022 | 0.003 |
| sp\|P68431\|H31_K9me3K14ac | 0.002 | 0.003 | 0.081 |

TABLE 11

| | CTL vs | SIRT6 WT vs | CTL vs |
|---|---|---|---|
| row ID | SIRT6cent | SIRT6cent | SIRT6 |
| sp\|P68431\|H31_K27K36K37 | 0.095 | 0.270 | 0.049 |
| sp\|P68431\|H31_K27K36me1K37 | 0.016 | 0.946 | 0.018 |
| sp\|P68431\|H31_K27K36me2K37 | 0.012 | 0.572 | 0.004 |
| sp\|P68431\|H31_K27me1K36K37 | 0.323 | 0.013 | 0.021 |
| sp\|P68431\|H31_K27me1K36me1K37 | 0.092 | 0.251 | 0.716 |
| sp\|P68431\|H31_K27me1K36me2K37 | 0.162 | 0.414 | 0.164 |
| sp\|P68431\|H31_K27me2K36K37 | 0.259 | 0.419 | 0.908 |
| sp\|P68431\|H31_K27me2K36me1K37 | 0.263 | 0.485 | 0.961 |
| sp\|P68431\|H31_K27me2K36me2K37 | 0.233 | 0.465 | 0.163 |
| sp\|P68431\|H31_K27me3K36K37 | 0.293 | 0.080 | 0.665 |
| sp\|P68431\|H31_K27me3K36me1K37 | 0.016 | 0.023 | 0.150 |

TABLE 12

| | CTL vs | SIRT6 WT vs | CTL vs |
|---|---|---|---|
| row ID | SIRT6cent | SIRT6cent | SIRT6 WT |
| sp\|P84243\|H33_K27K36K37 | 0.182 | 0.051 | 0.869 |
| sp\|P84243\|H33_K27K36me1K37 | 0.016 | 0.087 | 0.380 |
| sp\|P84243\|H33_K27K36me2K37 | 0.209 | 0.660 | 0.178 |
| sp\|P84243\|H33_K27acK36K37 | 0.027 | 0.140 | 0.422 |
| sp\|P84243\|H33_K27acK36me1K37 | 0.092 | 0.236 | 0.460 |
| sp\|P84243\|H33_K27acK36me2K37 | 0.360 | 0.423 | 0.632 |
| sp\|P84243\|H33_K27me1K36K37 | 0.718 | 0.375 | 0.387 |
| sp\|P84243\|H33_K27me1K36me1K37 | 0.789 | 0.244 | 0.191 |
| sp\|P84243\|H33_K27me1K36me2K37 | 0.260 | 0.648 | 0.318 |
| sp\|P84243\|H33_K27me2K36K37 | 0.041 | 0.252 | 0.239 |
| sp\|P84243\|H33_K27me3K36K37 | 0.909 | 0.979 | 0.850 |

Overexpression of SIR6 wt or SIRT6cent in 3T3-L1 led to changes in the acetylation profiles of histones H3.1, H3.2 and H4. This is reflected in significant difference between AdiWT and AdiCent at the levels of lysines (K) K5, K8, K12 and K16 in H4; K9, K18, K14 and K23 in H3.1; K27, K36 and K37 in H3.3. Hereby, H3K9, H3K18, H3K27, H3K23, H3K14, H3K36 histones were confirmed as SIRT6 targets, while H3K37, H4K5, H4K8 and H4K12 histones were identified as potentially new SIRT6 targets, not described in literature yet.

### Example 8: In Vivo Assays in HF/DEN Model

### Material and Methods

### Animal Models

36 male and 36 female of C57BL/6N-Tyr<cBrd>/BrdCrCrl Albino strain (Charles River) mice of 7-8 weeks old were included into the study. Animals were fed with high fat diet (HFD) (EF D12492, 60 kJ % Fat (Lard); ssniff Spezialdiäten GmbH—Germany) and 25 mg/kg DEN toxin or fed with control (CTL) diet (EF D12450B, 10 kJ % Fat (Lard/SBO); ssniff Spezialdiaten GmbH). All mice were weighted once a week, during the whole duration of the study. Seven weeks after HFD/DEN induction, male and female mice were randomly divided into 6 experimental groups, according to their sex: AAV-LUC (LUC), AAV-SIRT6wt (WT) and AAV-SIRT6cent (CEN), both for the CTL and HFD/DEN diet. Relative body weight gain, as compared to the individual body weight at the AAV-injection day were calculated. Areas under the curves were quantified. [0229]9 weeks after AAV treatment, mice were sacrificed and their organs harvested, weighted, and the relative organ weight normalized on the respective individual body weight was calculated.

### Hematological Evaluation

Hematological evaluation was performed on non-coagulable blood (with EDTA) and measured immediately using BC-2800 Vet (Mindray, Shenzhen, PRC) after blood bleeding/withdrawal from the axillary vessels during mice sacrifice by anesthetic overdose (xylazine 20 mg/kg+ketamine 300 mg/kg).

### Protein Expression Levels of SIRT6 and B-Catenin

Snap frozen liver tissue (up to 10 mg) was disintegrated in Ice-cold 1×RIPA buffer (supplemented with Halt™ Protease and Phosphatase Inhibitor Cocktail (100×), Thermofischer scientific) and kept on ice for 30 min with intervaled vortexing every 10 min with added sonication for 3×20 s. Tissue extracts were then centrifuged at 20000 g for 15 min at 4° C., supernatants were transferred to new tube and protein concentration was determined using a Pierce BCA assay (Pierce™ BCA Protein Assay Kit, Thermo Scientific). For the WB, 13 μg of the total protein per sample was used and loaded onto 10% stain-free polyacrylamide SDS-PAGE gels (TGX™ FastCast™ Acrylamide Kit, Biorad) and run for 200V for 35 min in cooled water bath. Proteins were then electro-transferred onto PVDF membranes using Trans-Blot Turbo RTA Mini 0.45 μm LF PVDF Transfer Kit (Bio-Rad) and Bio-Rad Trans-Blot Turbo Transfer System at 1.3 A and 25 V for 7 min. Membranes were then blocked with 5% v bovine serum albumin (BSA, P6154, BioWest) dissolved in TBST buffer (20 mM Tris-HCl, pH 7.6, 140 mM NaCl, 0.1% Tween 20) for at least 30 min. Next, the membranes were incubated overnight at 4° C. with primary antibody solutions: anti Vinculin (ab129002, rabbit mAb), anti SIRT6 (ab191385, EPR18463 rabbit) and anti B-catenin (8480S, rabbit mAB) (all in dilution 1:2000). Then, the appropriate blots were incubated with the horseradish peroxidase-conjugated anti-rabbit IgG secondary for at least 1 h at RT diluted 1:2500 in TBST blocking buffer. After three consecutive washes with TBST, protein levels were detected by Clarity™ Western ECL Substrate (1705060, Bio-Rad), and the signal detected on Bio-Rad ChemiDoc XRS+imaging systems. For quantitative measurement, the scanned membranes were analyzed using the Image Lab™ Software (Bio-Rad). Expression/abundance of SIRT6 and B-catenin was normalized to Vinculin.

### Biodistribution

In vivo bioluminescence imaging (BLI) allows repeated assessment of reporter gene expression in tissues of living mice injected with suitable viral vector throughout the course of experiment, without the need to sacrifice animals. With a sensitive reporter gene (e.g., Luc2), BLI permits estimated localization of vector-infected tissues and quantification of their expression levels. Hence, we used the AAV8.CMV-Luc2 vector with identical dosing schemes to that of the AAV.CMV-SIRT6cent vector. Different dosing schemes were used in group E (2.5×10\hat{ }12 vg/kg (medium dose), 2 injections); group F (2.5×10\hat{ }12 vg/kg (medium dose), 1 injection), group G (1.0×10\hat{ }13 vg/kg (high dose), 2 injections); group H (1.0×10\hat{ }13 vg/kg (high dose), 1 injection) with 5 mice per group. LUC2 expression was followed in 20 mice with in vivo imaging system IVIS Lumina II twice a week starting from Day 7 up to Day 28 after viral vector injection.

AAV8.CMV-SIRT6c vector expression in mouse tissues was analyzed 28 days post injection by reverse transcription-quantitative PCR (qPCR). Total RNA was isolated from tissues and subjected to reverse transcription using oligo-dT(20) primer. WPRE sequence was chosen as qPCR target sequence, since it is present in the mature AAV-vector derived mRNA (in both SIRT6c and Luc2 vectors) and there are no homologous sequences in the mouse genome or transcriptome. For absolute quantification different dilutions of SIRT6c-WPRE plasmid with known copy number was used as a reference against which the samples Ct values were converted into copy number per nanogram of total RNA.

Relative SIRT6 protein expression, compared to b-tubulin, was assessed in tissue organs of mice 28 days after treatment with AAV-SIRT6c or AAV-Luc, at medium and high doses, by western blot.

### Results

### Mice Weight

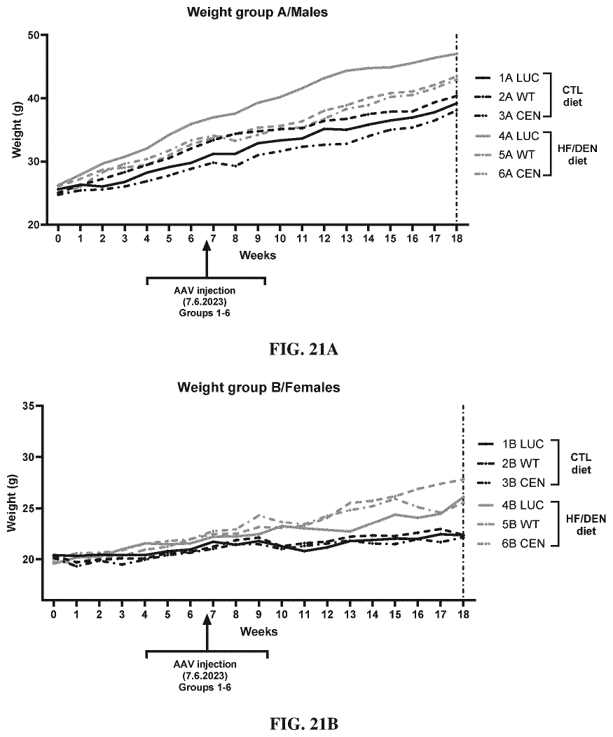

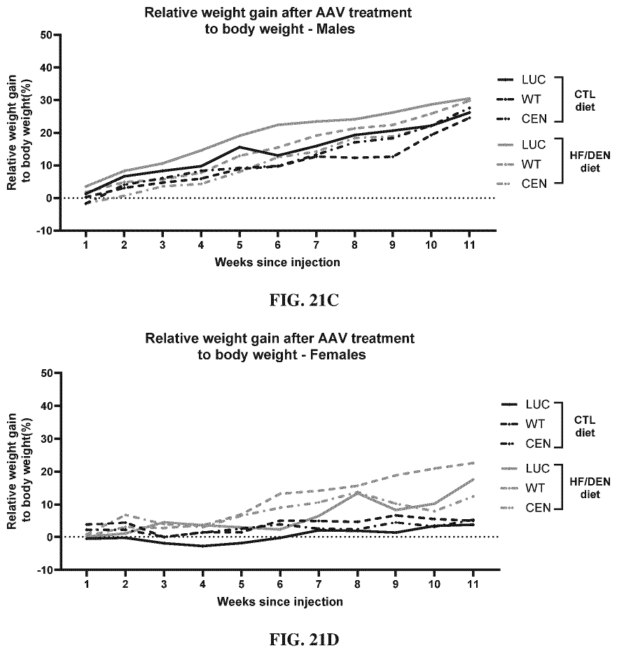

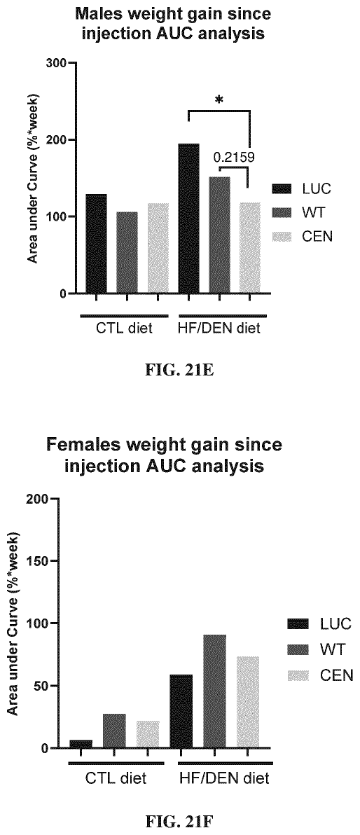

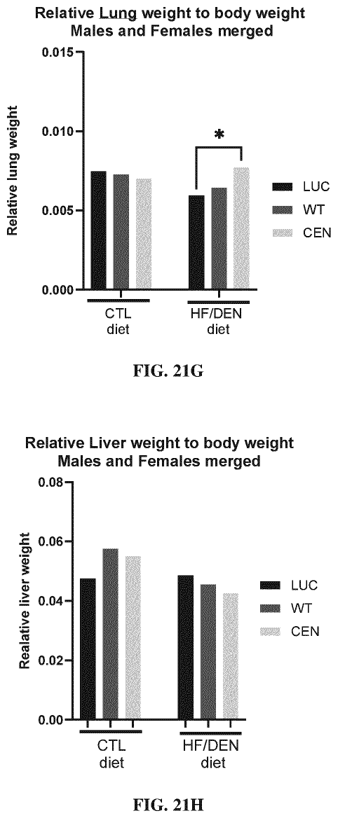

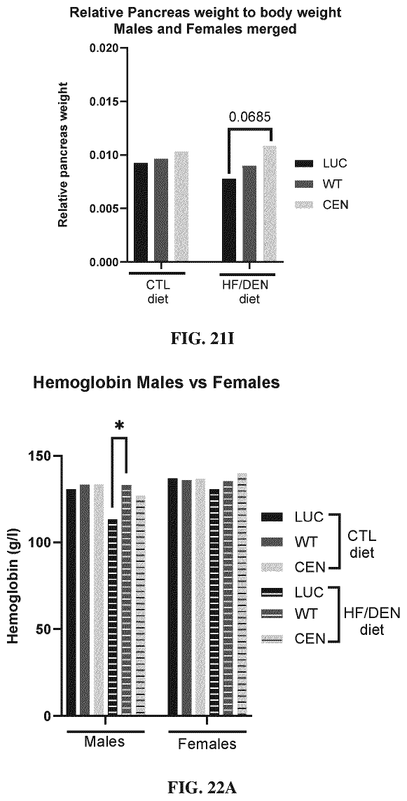

While no differences were observed for female mice, weight and relative weight gain is decreased for male mice that received HFD/DEN diet and were treated with AAV-SIRT6wt or AAV-SIRT6cent compared to mice that received the same diet but were treated with AAV-LUC instead (FIG. 21A-21F). This was also seen in the area under the curve quantification of the relative weight gain.

### Organ Weight

Mice that received control diet and were treated with AAV-LUC, AAV-SIRT6wt or AAV-SIRT6cent no differences could be observed (FIG. 21G-21I). For Mice induced with HFD/DEN diet and treated with AAV-SIRT6cent, increased lung and pancreas weight could be observed compared to Mice that received the same HFD/DEN diet but were treated with AAV-SIRT6wt and AAV-LUC instead. More important, Mice induced with HFD/DEN diet and treated with AAV-SIRT6cent showed decreased liver weight compared to the other treatment groups.

Haematological Examination in Males and Females from HFD/DEN Model

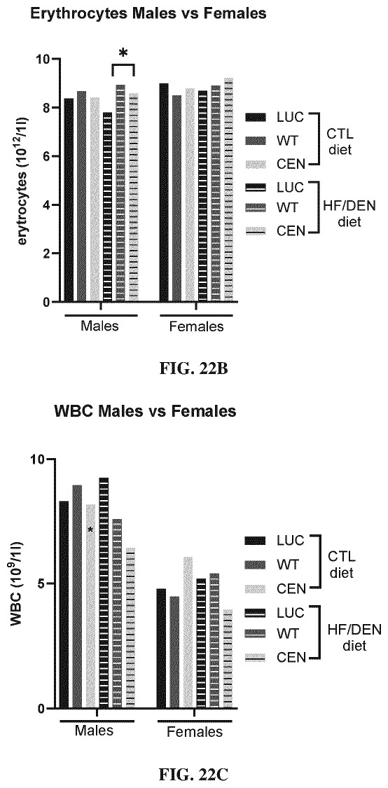

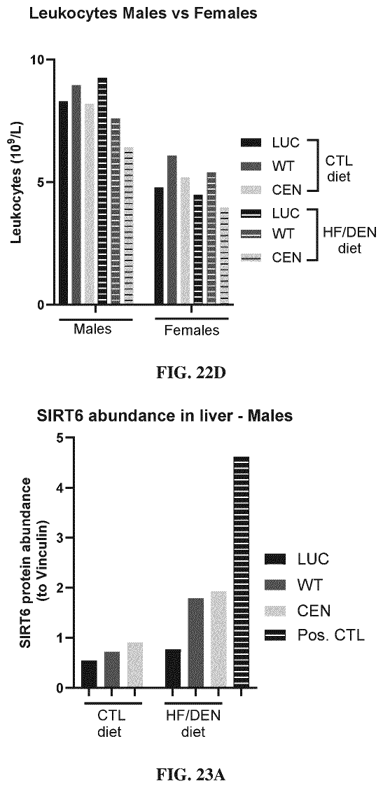

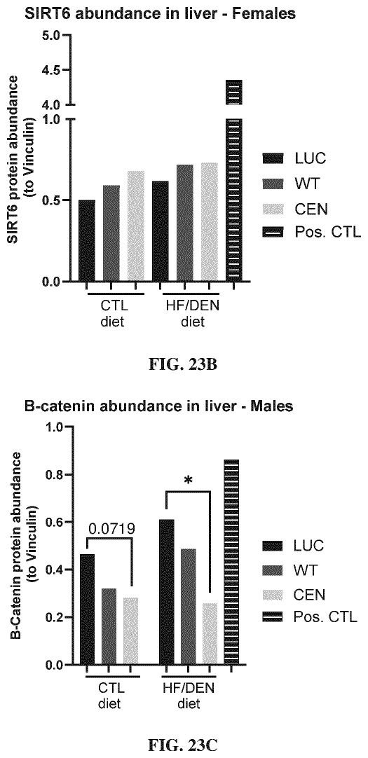

Within each blood parameter males and females are shown for mice that received control versus HFD/DEN induction (FIG. 22A-22D). Treatment with AAV-SIRT6wt and AAV-SIRT6cent resulted in higher hemoglobin and erythrocytes levels within male mice induced with HFD/DEN diet, while white blood cells (WBC) and leukocytes are reduced in the same treatment groups.

### Protein Expression Levels of SIRT6 and B-Catenin

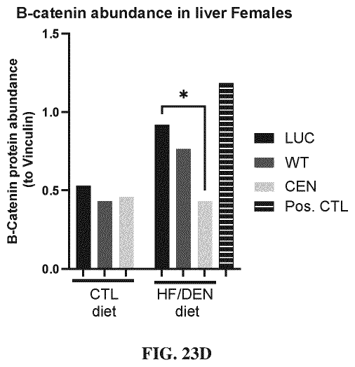

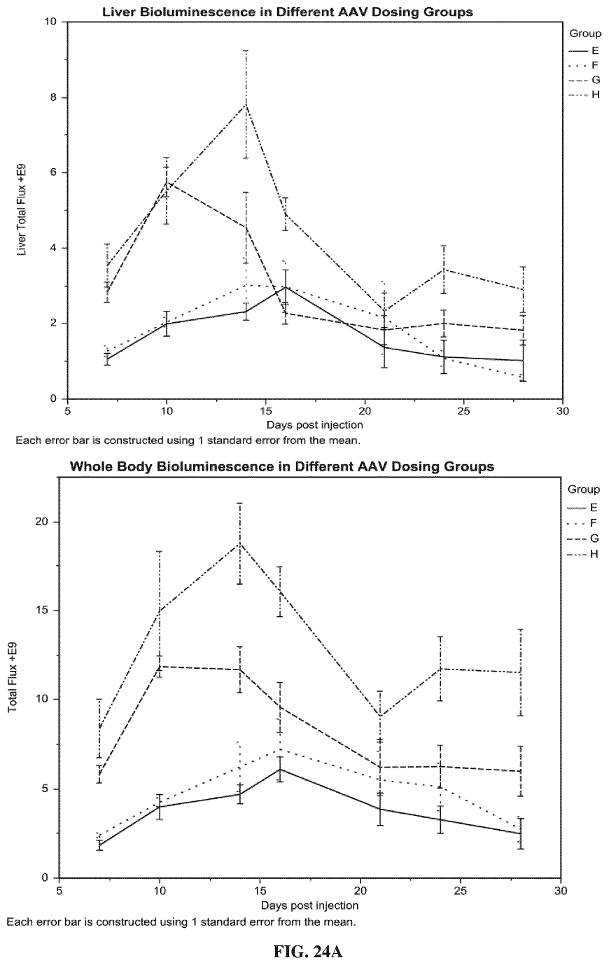

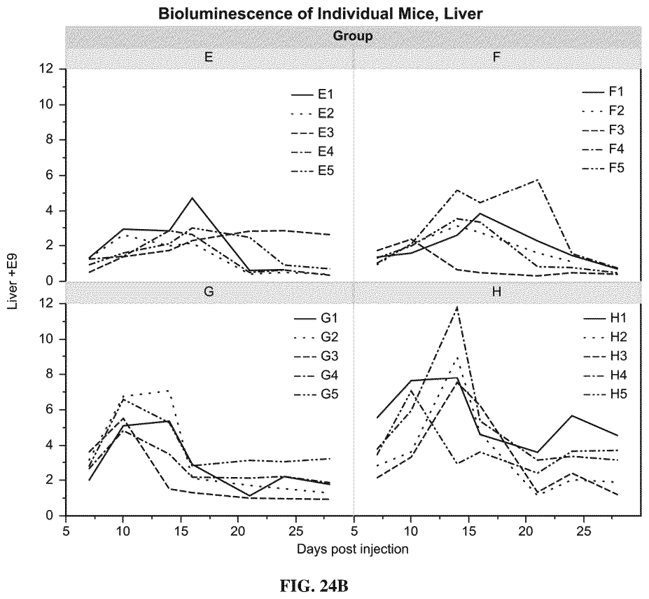

Results are shown in FIG. 23A-23D.

Western blot analysis showed that SIRT6 levels were clearly increased for AAV-SIRT6wt and AAV-SIRT6cent 9 weeks after AAV injection in HFD-DEN treated groups in male and female mice.

By treating these mice with AAV-SIRT6wt and AAV-SIRT6cent B-catenin levels reduced both in males and females. These observations were more pronounced for AAV-SIRT6cent treated animals that received HFD/DEN diet.

### Reporter (LUC) Biodistribution

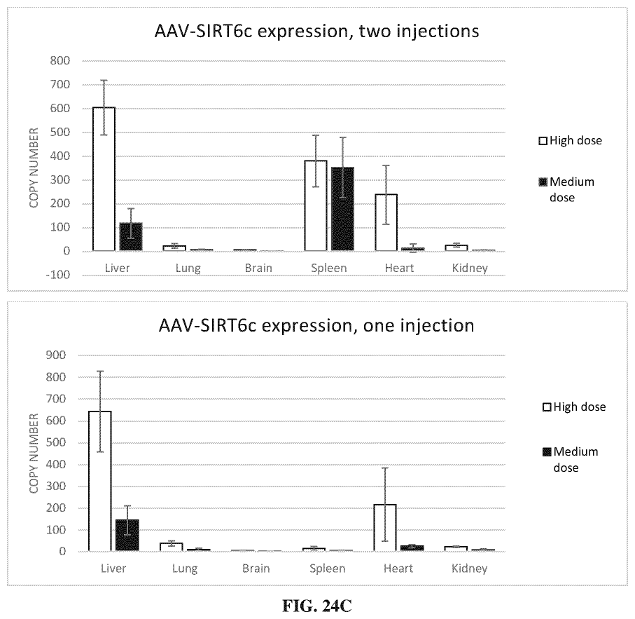

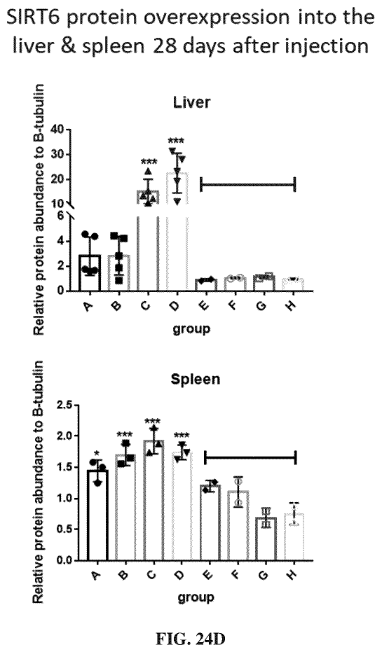

BLI analysis showed that tail vein injection of AAV Luc2 vector was 100% successful—all of 20 mice yielded BLI signal (FIG. 24A-24B). Further, the highest levels of signal were recorded between 10-16 days postinjection, both in the liver anatomical region and within the whole body, and the signals were in accordance with the vector doses. After that peak, the overall BLI signal was gradually decreasing until the end of 28-day period, but the levels remained higher in mice that had received higher vector doses.

### SIRT6c Biodistribution

28 days after 2 injections of AAV-SIRT6c at high dose, SIRT6c mRNA expression was found mainly into the liver, and, at a lower level, in spleen and heart (FIG. 24C). However, at medium dose, a lower SIRT6c expression was found into the liver, while similar expression was detected into the spleen (top panel). Minor expression was found into the heart.

28 days after a single injection of AAV-SIRT6c at high dose, SIRT6c mRNA expression was found mainly into the liver, and at a lower level into the heart of treated mice (FIG. 24C). After single medium dose injection, SIRT6c expression was found mainly into the liver, at lower levels compared to high dose (bottom panel).

SIRT6c protein overexpression was pronounced into the liver and at minor levels detected into the spleen of AAV-SIRT6c treated mice at high and medium doses, 28 days after treatment compared to AAV-LUC treated control mice, as shown by western blot (FIG. 24D).

1.-21. (canceled)

22. A method of preventing and/or treating non-alcoholic fatty liver disease (NAFLD) comprising administering to a patient in need thereof a therapeutically effective amount of an isolated nucleic acid molecule encoding a variant of sirtuin 6 (SIRT6) having at least 75% identity with sequence SEQ ID NO: 1, the variant having at least one mutation selected in the group comprising or consisting of a substitution N308K and a substitution A313S with respect to sequence SEQ ID NO: 1, or of an isolated polypeptide encoded by the same, or of a pharmaceutical composition comprising the same.

23. The method according to claim 22, wherein the isolated nucleic acid molecule has a sequence selected in the group comprising or consisting of SEQ ID NO: 6, SEQ ID NO: 7 and SEQ ID NO: 8.

24. The method according to claim 22, wherein the isolated polypeptide encoded by the isolated nucleic acid molecule has a sequence selected in the group comprising or consisting of SEQ ID NO: 2, SEQ ID NO: 3 and SEQ ID NO: 4.

25. The method according to claim 22, wherein the NAFLD is Stage 1 NAFLD or 2 NAFLD.

26. The method according to claim 25, wherein the NAFLD is Stage 1 NAFLD.

27. The method according to claim 25, wherein the NAFLD is Stage 2 NAFLD.

28. The method according to claim 22, wherein the NAFLD is non-alcoholic steatohepatitis (NASH) Stage 1.

29. The method according to claim 22, wherein the NAFLD is non-alcoholic steatohepatitis (NASH) Stage 2.

30. The method according to claim 22, wherein the NAFLD is non-alcoholic steatohepatitis (NASH) Stage 3.

31. The method according to claim 22, wherein the isolated nucleic acid molecule is comprised in a vector, preferably a viral vector, more preferably an adeno-associated viral vector (AAV), an exosome-associated AAV vector (exo-AAV), an exosome, an adenoviral vector, a retroviral vector, or a herpes virus vector.

32. The method according to claim 31, wherein the vector is a viral vector.

33. The method according to claim 32, wherein the viral vector is an adeno-associated viral vector (AAV), an exosome-associated AAV vector (exo-AAV), an exosome, an adenoviral vector, a retroviral vector, or a herpes virus vector.

34. The method according to claim 22, wherein the isolated nucleic acid molecule is comprised in a suspension.

35. The method according to claim 31, wherein the vector is comprised in a suspension.

36. The method according to claim 22, wherein the isolated nucleic acid molecule, or the isolated polypeptide encoded by the same, or the pharmaceutical composition comprising the same, is comprised in a cell.

37. The method according to claim 22, wherein the pharmaceutical composition comprising the isolated nucleic acid molecule further comprises a pharmaceutically acceptable excipient.

38. The method according to claim 22, further comprising administering to the patient another therapeutic agent.


SEQUENCE LISTING (21 sequences)

SEQ ID NO: 1 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 2 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRIKGSIPAGPKQEPCAQHNGSEPASPKRERPTSPAPHRPPKRVKAKAVPS

SEQ ID NO: 3 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPSGPKQEPCAQHNGSEPASPKRERPTSPAPHRPPKRVKAKAVPS

SEQ ID NO: 4 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRIKGSIPSGPKQEPCAQHNGSEPASPKRERPTSPAPHRPPKRVKAKAVPS

SEQ ID NO: 5 - DNA, 1068 bp, Homo sapiens
  source 1..1068: mol_type = other DNA
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaagggctctatccccgccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 6 - DNA, 1068 bp, Homo sapiens; identical to SEQ ID NO: 5
  source 1..1068: mol_type = other DNA

SEQ ID NO: 7 - DNA, 1068 bp, Homo sapiens
  source 1..1068: mol_type = other DNA
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatcccctccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 8 - DNA, 1068 bp, Homo sapiens
  source 1..1068: mol_type = other DNA
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaagggctctatcccctccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 9 - DNA, 21 bp, Synthetic construct
  source 1..21: mol_type = other DNA
aaaagacagctacgtgggtga

SEQ ID NO: 10 - DNA, 22 bp, Synthetic construct
  source 1..22: mol_type = other DNA
gccatgttctatcgggtacttc

SEQ ID NO: 11 - DNA, 21 bp, Synthetic construct
  source 1..21: mol_type = other DNA
gtgcgatgacgtgatctgtga

SEQ ID NO: 12 - DNA, 19 bp, Synthetic construct
  source 1..19: mol_type = other DNA
cggtggtttcttggtcggt

SEQ ID NO: 13 - DNA, 23 bp, Synthetic construct
  source 1..23: mol_type = other DNA
accaccttataccagcgttatga

SEQ ID NO: 14 - DNA, 21 bp, Synthetic construct
  source 1..21: mol_type = other DNA
ggtgtagacgaaccggatgtc

SEQ ID NO: 15 - DNA, 21 bp, Synthetic construct
  source 1..21: mol_type = other DNA
agtccactgagtaccggagac

SEQ ID NO: 16 - DNA, 22 bp, Synthetic construct
  source 1..22: mol_type = other DNA
catttcacgcaatctggcgttc

SEQ ID NO: 17 - DNA, 23 bp, Synthetic construct
  source 1..23: mol_type = other DNA
tacaggatcattggctacacacc

SEQ ID NO: 18 - DNA, 19 bp, Synthetic construct
  source 1..19: mol_type = other DNA
ggtcacatcgctccagact

SEQ ID NO: 19 - DNA, 17 bp, Synthetic construct
  source 1..17: mol_type = other DNA
ggtgcgtgcccagttga

SEQ ID NO: 20 - DNA, 19 bp, Synthetic construct
  source 1..19: mol_type = other DNA
tactttctccccgcttttt

SEQ ID NO: 21 - DNA, 1068 bp, Homo sapiens
  source 1..1068: mol_type = genomic DNA
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctga